(19) United States
(12) Patent Application Publication
(10) Pub. No.: US 2022/0031745 A1
(43) Pub. Date: Feb. 3, 2022

(54) ANTIGEN DENSITY SENSING MOLECULAR CIRCUITS AND METHODS OF USE THEREOF

(71) Applicant: The Regents of the University of California, Oakland, CA (US)

(72) Inventors: Wendell A. Lim, San Francisco, CA (US); Rogelio A. Hernandez-Lopez, San Francisco, CA (US)

(21) Appl. No.: 17/278,628

(22) PCT Filed: Sep. 26, 2019
(86) PCT No.: PCT/US2019/053151

§ 371 Date: Mar. 22, 2021

Related U.S. Application Data
(60) Provisional application No. 62/738,995, filed on Sep. 28, 2018.

(51) Int. Cl. A61K 35/17; A61P 35/00; C12N 5/0783

(52) U.S. Cl. CPC A61K 35/17; C12N 5/0636; A61P 35/00

(57) ABSTRACT

Provided are antigen-density sensing molecular circuits and methods of using the same. Aspects of such circuits will generally include an antigen-triggered switch component and a therapeutic component specific for the same antigen as the antigen-triggered switch component. The circuits will generally be configured such that expression of the therapeutic component is induced by the antigen-triggered switch component when the switch is activated by binding the antigen. Nucleic acids, expression constructs, vectors and the like encoding such circuits, and cells genetically modified to include an antigen-density sensing molecular circuit are also provided. Also provided are methods of making antigen-density sensing molecular circuits, methods of inducing expression of high affinity therapeutics specific to an antigen expressed by a target cell, methods of activating an immune response to a target cell, methods of treating a subject for a cancer expressing an antigen, and the like, where such methods involve antigen-density sensing molecular circuits.

### CROSS-REFERENCE

This application claims the benefit of U.S. Provisional Patent Application No. 62/738,995, filed Sep. 28, 2018, which application is incorporated herein by reference in its entirety.

### INCORPORATION BY REFERENCE OF SEQUENCE LISTING PROVIDED AS A TEXT FILE

A Sequence Listing is provided herewith as a text file, “UCSF-575WO_SEQ_LISTING_ST25.txt” created on Sep. 20, 2019 and having a size of 11 KB. The contents of the text file are incorporated by reference herein in their entirety.

### INTRODUCTION

T cells can be redirected to kill tumor cells via synthetic T cell receptors known as chimeric antigen receptors (CARs), this approach is becoming a highly promising therapeutic strategy for cancer treatment. CAR T cell therapies for the treatment of cancer have even resulted in clearance for some patients and such therapies are gaining widespread adoption and approval, including by regulatory agencies such as the Food and Drug Administration.

Despite recent successes using CAR T cell therapies, engineered CAR T cells face many obstacles to be applied more broadly to solid tumors and to increase their safety. A lack of truly cancer specific antigens results in off-target effects in some tissues. Such off-target effects can be amplified by the immense immune activating power of this new class of therapies, in some cases leading to lethal side effects associated with T cell attack of normal tissues.

### SUMMARY

Provided are antigen-density sensing molecular circuits and methods of using antigen-density sensing molecular circuits. Aspects of such circuits will generally include an antigen-triggered switch component, such as an antigen-triggered transcriptional switch, and a therapeutic component that is specific for the same antigen as the antigen-triggered switch component. The circuits of the present disclosure will generally be configured such that expression of the therapeutic component is induced by the antigen-triggered switch component when the switch is activated by binding the antigen.

Nucleic acids, expression constructs, vectors and the like encoding such circuits as well as cells genetically modified to include an antigen-density sensing molecular circuit are also provided. Also provided are methods of making antigen-density sensing molecular circuits, methods of inducing expression of high affinity therapeutics specific to an antigen expressed by a target cell, methods of activating an immune response to a target cell, methods of treating a subject for a cancer expressing an antigen, and the like, where such methods involve antigen-density sensing molecular circuits.

### BRIEF DESCRIPTION OF THE DRAWINGS

FIGS. 1A-1B provide a schematic depiction of bystander cells and tumor cells having varying levels of antigen density. (FIG. 1A) Schematic depiction of CAR T cells with varying level of antigen density. (FIG. 1B) Schematic depiction of common mechanisms for ultrasensitive sensing.

FIG. 2 depicts the current strategy for chimeric antigen receptor (CAR) immune cell activation using linear antigen recognition, resulting in death of both high antigen density tumor cells and bystander cells with lower antigen density.

FIG. 3 depicts a strategy for CAR immune cell activation based on antigen-density sensing and cooperative recognition, resulting in death of high antigen density tumor cells and survival of bystander cells with lower antigen density.

FIG. 4 schematically depicts the tunable recognition of antigen-density sensing CAR circuits.

FIG. 5 provides a schematic depiction of one embodiment of an antigen-density sensing circuit of the present disclosure, and the activation thereof by an antigen.

FIG. 6 depicts embodiments employing varied antigen binding domain valency for cooperative antigen affinity, using an anti-Her2 CAR as a non-limiting example.

FIG. 7A-7D provide the design of engineered T cells employing a synNotch-CAR circuit for the recognition and discrimination of Her2 expressing tumor cells from Her2 expressing bystander cells based on Her2 antigen density sensing according to an embodiment of the present disclosure.

FIGS. 8A-8D provide a schematic design of a construct encoding an anti-Her2 CAR. (FIG. 8A) Design of anti-Her2 CAR used in this study. (FIG. 8B) Effect of changing CAR expression levels on antigen density dependent cell killing. (FIG. 8C) Effect of changing CAR affinity on antigen density dependent cell killing. (FIG. 8D) Changing CAR affinity or expression leads to linear changes in antigen density response curves.

FIG. 9 demonstrates the construction of Her2 cells expressing the Her2 antigen at various antigen densities and associated quantification thereof.

FIGS. 10A-10E provide a schematic design of a construct encoding and anti-Her2 synNotch (top) and a construct encoding a fluorescently tagged anti-Her2 CAR used in a synNotch-CAR circuit according to an embodiment described herein. (FIG. 10A) Design of two-step CAR T circuit. (FIG. 10B) To track CAR expression, a mCherry protein was fused to the C-terminus of the anti-Her2 CAR construct. (FIG. 10C) In vitro cell killing curve as a function of target cell antigen density. (FIGS. 10D-10E) FACS distributions and quantitation for CAR expression and T cell proliferation measured as a function of target cell Her2 density (at 3 days) for the circuit T cells.

FIG. 11 depicts the expression of Her2 synNotch and CAR constructs employed in a circuit as described herein.

FIG. 12 demonstrates that low affinity SynNotch receptors gate CAR expression in an antigen density dependent manner.

FIG. 13 demonstrates that affinity tuned SynNotch-CAR circuits discriminate between cells with different antigen levels to differentially kill target cells with high antigen density.

FIG. 14 provides the levels of CAR expression and immune cell activation by cells expressing CARs of differing affinity as compared to a synNotch-CAR circuit described herein when such cells are exposed to various different antigen densities.

FIG. 15 demonstrates that a low affinity Her2 CAR does not discriminate between low and high antigen density targets.

FIG. 16 demonstrates that a high affinity Her2 CAR does not discriminate between low and high antigen density targets.

FIG. 17 demonstrates that a SynNotch-CAR circuit is capable of discriminating between low and high antigen density targets.

FIGS. 18A-18D provide a schematic depiction of a two tumor mouse model, and the treatment regimen thereof, used to test the antigen-density sensing circuits described herein. (FIG. 18A) In vitro target cell area over time: (top plot) Low Her2 density cancer cells, PC3 (1+ tumor line), or (bottom plot) High Her2 density cancer cells, SKOV3 (3+ tumor line). (FIG. 18B) Representative images of the in vitro cell killing experiment. (FIG. 18C) Representative images of the in vitro cell killing experiment for the T cells expressing a two-step circuit (low affinity to medium affinity CAR). (FIG. 18D) Schematics of a two tumor mouse model experiment to test the efficacy and safety of ultrasensitive antigen density sensing T cells.

FIG. 19 shows that high affinity CAR T cells did not discriminate between high and low antigen density tumors, reducing the tumor volume in both right and left flank tumors.

FIG. 20 shows that synNotch-CAR circuit CAR T cells discriminated between high and low antigen density tumors in vivo, reducing the tumor volume in high antigen density tumors while low antigen density tumors increased in volume similar to untransduced controls. The solid lines show the mean and the error bars the standard error of the mean (n=7).

FIGS. 21A-21C provide determination of antigen density and receptor expression from fluorescence intensity. Antigen density and receptor expression were determined by quantitative flow cytometry. (FIG. 21A) Representative flow cytometry histograms showing the fluorescence intensity of Quantum Symply Cellular anti-Mouse IgG beads (Bang Laboratories 815) stained with anti-Her2 APC antibody. (FIG. 21B) Engineered T cells expressing either a constitutive CAR or SynNotch receptor were stained with anti-myc Alexa 647. The number of receptors per T cell populations was determined as described above. (FIG. 21C) Representative flow cytometry histograms of beads showing fluorescence intensity equivalent to the indicated number of soluble mCherry molecules (MESF).

FIGS. 22A-22E provide killing assay gating scheme, CAR T cell receptor expression and trogocytosis analysis. (FIG. 22A) Details on gating scheme utilized to analyze killing assays by flow cytometry. (FIG. 22B) Construct design to obtain low expression levels of anti-Her2 CARs. C. T cell CAR expression levels as a function of target antigen density after 3 days of co-culture. (FIG. 22D) Ratio of T cell counts when cultured either alone or with K562-Her2 targets after 3-days of co-culture. (FIG. 22E) Representative FACS histograms of BFP fluorescence intensity shown by T cells after 3 days of culture with K562-Her2 (BFP-tagged) targets.

FIGS. 23A-23E show effects of receptor affinity and T cells dosage on two-step circuit function. (FIG. 23A) Four parameter Hill equation utilized to fit the killing response curves as a function of antigen density of two-step circuits tested in this study (FIG. 23B) Target cell killing response curves for T cells expressing other two-step circuits. (FIG. 23C) Target cell killing response curves for T cells expressing low affinity SynNotch to medium affinity CAR circuit at different effector to target (E:T) ratios. (FIG. 23D) Target cell killing response curves for T cells expressing two-step circuits where the SynNotch affinity was changed. (FIG. 23E) Target cell killing response curve for T cells expressing low affinity SynNotch to medium affinity CAR circuit from a different donor.

FIGS. 24A-24E show T cells expressing a two-step circuit low-to-high SynNotch-CAR affinity recognition circuit yield ultrasensitive antigen density sensing against EGFR engineered cells. (FIG. 24A) Representative flow cytometry histograms showing the fluorescence intensity of Quantum Symply Cellular anti-Mouse IgG beads (Bang Laboratories 815) stained with anti-EGFR BV786 antibody. (FIG. 24B) Representative flow cytometry histograms of engineered K562 EGFR cell lines stained with anti-EGFR BV786 antibody. (FIG. 24C) Series of ScFv and nanobodies utilized to build two-step SynNotch to CAR circuits. Their reported affinities are indicated. (FIG. 24D) Target cell killing activity as a function of EGFR antigen density for T cells expressing CARs of indicated affinities. (FIG. 24E) Target cell killing activity as a function of EGFR antigen density for T cells expressing a low affinity SynNotch to high affinity CAR circuit.

FIGS. 25A-25B show that low affinity SynNotch to medium affinity CAR T cells show antigen density activity against several Her2 positive cancer cell lines. (FIG. 25A) In vitro target cell area over time (FIG. 25B) Representative FACS plots of inducible CAR expression and T cell proliferation for T cells co-cultured with cancer cell lines expressing high and low Her2 densities.

FIG. 26 shows tumor volume measurements for individual mice treated with T cells expressing low affinity SynNotch to medium affinity CAR circuit.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein, refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 108 M, 5×10⁻⁸ M, 10′ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ M, 10⁻⁵ M, 10⁻⁴ M). In some contexts, e.g., binding between an antigen binding domain or a macromolecule containing one or more antigen binding domains and antigen(s), “specific binding” can be in the range of from 1 nM to 100 nM, 1 μM to 100 μM, or from 100 μM to 1 mM.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland) and the anti-CD19-CD28-CD3ζ CAR of Axicabtagene Ciloleucel as commercialized by Kite Pharma, Inc. (Santa Monica, Calif.).

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8′ T cells, natural-killer (NK) cells, and neutrophils, which cells are capable of mediating cytotoxicity responses.

The term “synthetic” as used herein generally refers to an artificially derived polypeptide or polypeptide encoding nucleic acid that is not naturally occurring. Such synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “bystander cell”, as used herein generally describes cells that are not intentionally targeted by a therapeutic or a therapeutic expressing cell. Bystander cells may, in some instances, express the same antigen as a targeted cell type, where a “targeted cell type” refers to the cell type that is intentionally targeted by a therapeutic or therapeutic expressing cell. Accordingly, in conventional therapies bystander cells may, in some instances, be unintentionally targeted by an antigen specific therapeutic or an antigen-targeted therapeutic cell due to expression of the target antigen by the bystander cell. Bystander cells may be of, reside in, or be derived from essentially any human tissue (e.g., connective tissue, muscular tissue, nervous tissue, epithelial tissue, blood tissue, bone tissue, tendon tissue, ligament, adipose tissue, areolar tissue, fibrous connective tissue, skeletal connective tissue, fluid connective tissue, visceral/smooth muscle, skeletal muscle, cardiac muscle, central nervous system tissues, peripheral nervous system tissues, simple squamous epithelium, stratified squamous epithelium, simple cuboidal epithelium, transitional epithelium, pseudostratified columnar epithelium, columnar epithelium, glandular epithelium, ciliated columnar epithelium, and the like). In some instances, a target cell may be derived from the same tissue as a bystander cell, including but not limited to e.g., where a target cancer cell is derived from a tissue that includes non-cancerous bystander cells.

The term “antigen density threshold”, as used herein generally refers to a concentration of antigen expressed by a cell at or above which an antigen-density sensing circuit of the present disclosure is activated. By “activated” in this context is generally meant that the components of the molecular circuit are activated and/or expressed resulting in the output of the circuit. For example, where the circuit-containing cell is an immune cell and the output of the circuit is immune activation, interaction of the circuit-containing cell with a cell having an antigen-density above the antigen density threshold will cause immune activation of the circuit-containing cell. Correspondingly, where the circuit-containing cell is an immune cell and the output of the circuit is immune activation, interaction of the circuit-containing cell with a cell having an antigen-density below the antigen density threshold will not cause immune activation of the circuit-containing cell. The antigen density threshold of a circuit may be set based on the relative affinities of components of the circuit for an antigen to which the circuit responds. Correspondingly, antigen density threshold of a circuit may be modified by modifying the relative affinities of components of the circuit for the antigen. Antigen density thresholds may be expressed in relative terms (e.g., one circuit may have an antigen density threshold that his higher or lower than another circuit) or absolute terms (e.g., a circuit may have an antigen density threshold of X unit of antigen per cell (e.g., molecules/cell).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides antigen-density sensing molecular circuits and methods of making and using antigen-density sensing molecular circuits. The term “antigen-density sensing” generally refers to the ability of a system or a cell to produce a particular response based on the density of a particular antigen encountered by the system or cell, e.g., as expressed by a target cell. For example, where the encountered antigen-density is relatively high the system or cell may generate one response through the circuit and where the encountered antigen-density is low the system or cell may generate a second response or no response.

As an example, an antigen-density sensing molecular circuit of the present disclosure may or may not drive the expression of an encoded therapeutic based on the encountered antigen density, including e.g., where the circuit drives expression of the encoded therapeutic when a relatively high antigen-density is encountered and the circuit does not drive expression of the encoded therapeutic when a relatively low antigen-density is encountered. In some instances, an antigen-density sensing molecular circuit of the present disclosure may modulate the level of expression of an encoded therapeutic based on the encountered antigen density, including e.g., where the circuit induces an increased level of expression of the encoded therapeutic when a relatively high antigen-density is encountered and the circuit does not induce an increased level of expression of the encoded therapeutic when a relatively low antigen-density is encountered. Such circuits will vary, as described in more detail below, and such circuits find use in a variety of methods, as also described in more detail below.

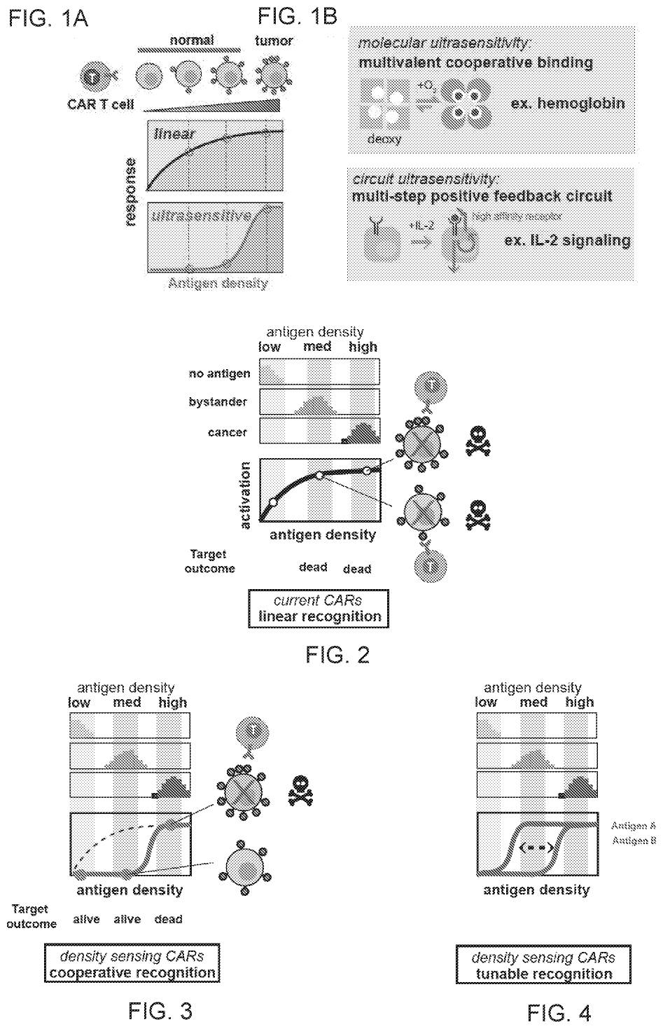

Antigen-density sensing molecular circuits may allow for the discrimination between cells that express a low amount of a particular antigen and cells that express a high amount of the antigen. For example, as depicted in FIG. 1, an antigen that is expressed at a high level on tumor cells (antigen density high) may also be expressed at lower levels by bystander cells (antigen density low). Thus, without antigen-density sensing, a therapeutic directed to the antigen will target, and e.g., kill, the tumor cells as well as the bystander cells. This is the case for the linear antigen recognition employed by current chimeric antigen receptor (CAR) therapies. For example, as schematized in FIG. 2, both tumor cells having high antigen density and bystander cells having low antigen density induce sufficient CAR T cell activation to result in death of both tumor cells and bystander cells.

The circuits and methods of the present disclosure apply cooperative (FIG. 3) and tunable (FIG. 4) recognition through antigen-density sensing. Thus, as schematized in FIG. 3, whereas high antigen-density tumor cells activate and are killed by CAR T cells, low antigen-density bystander cells do not sufficiently activate the CAR T cells and thus the bystander cells are not targeted for killing. In addition, as schematized in FIG. 4, depending on the level(s) of antigen density expressed by target cells and/or bystander cells, the threshold for recognition and CAR-mediated killing can be tuned, e.g., by adjusting the relative affinity of the antigen binding domains employed.

### Circuits

As summarized above, the present disclosure provides circuits, also referred to in some instances as molecular circuits. Such circuits may be encoded by nucleic acid sequences and may, in some instances, be present and/or configured in expression vectors and/or expression cassettes. The subject nucleic acids of the present circuits may, in some instances, be contained within a vector, including e.g., viral and non-viral vectors. Such circuits may, in some instances, be present in cells, such as immune cells, or may be introduced into cells by various means, including e.g., through the use of a viral vector. Cells may, in some instances, be genetically modified to encode a subject circuit, where such modification may be effectively permanent (e.g., integrated) or transient as desired.

Encoded components of the circuits of the present disclosure will generally include at a minimum at least one encoded antigen-triggered transcriptional switch and at least one encoded antigen-specific therapeutic. Circuits of the present disclosure sense the density of a single type of antigen encountered, e.g., by a cell expressing circuit components. Accordingly, the output or response of a cell containing a molecular circuit of the present disclosure will be dependent on the density of the antigen encountered by the cell, where e.g., encountering high antigen-density will cause the cell to express the encoded antigen-specific therapeutic whereas encountering low antigen-density will cause the cell to either not express the encoded antigen-specific therapeutic or express the encoded antigen-specific therapeutic at an insignificant level. In some instances, such molecular circuits allow a cell, e.g., a therapeutic cell, to produce a particular response, e.g., expression of an effective amount of therapeutic, only when antigen density above a particular threshold is encountered.

Aspects of such circuits will generally include an antigen-triggered switch component, such as an antigen-triggered transcriptional switch, and a therapeutic component that is specific for the same antigen as the antigen-triggered switch component. The circuits of the present disclosure will generally be configured such that expression of the therapeutic component is induced by the antigen-triggered switch component when the switch is activated by binding the antigen.

Antigen-density sensing in such circuits may be achieved through the use of different antigen binding domains, having different affinity for the same antigen, on components of the circuit. For example, the antigen-triggered switch component may employ a first antigen binding domain for the antigen that is of low affinity and the therapeutic may employ a second antigen binding domain for the antigen that is of high affinity. Antigen binding domains having different affinities for an antigen may or may not be derived from the same antigen binding domain or antigen binding macromolecule. For example, in some instances, two antigen binding domains having different affinities for the same antigen may be derived from the same antigen binding domain, including e.g., where one is a modified version or variant of the other. In some instances, two antigen binding domains having different affinities for the same antigen may be derived from different antigen binding domains, including e.g., antigen binding domains derived from different antibodies to the same antigen. Antigen binding domains having different affinities for the same antigen may or may not bind the same epitope on the antigen.

Affinity may be expressed in relative or absolute terms. Accordingly, the affinity of an antigen binding domain or a macromolecule having one or multiple antigen binding domains may be referred to as low, e.g., as compared to an antigen binding domain or a macromolecule having a higher affinity, or reduced, e.g., as compared to an antigen binding domain or a macromolecule from which it was derived, and the like. In addition, the affinity of an antigen binding domain or a macromolecule having one or multiple antigen binding domains may be referred to as high, e.g., as compared to an antigen binding domain or a macromolecule having a lower affinity, or enhanced, e.g., as compared to an antigen binding domain or a macromolecule from which it was derived, and the like. In some instances, affinity may be expressed for a particular antigen binding domain or a macromolecule having one or multiple antigen binding domains in terms of a dissociation constant (Kd), such as is described in more detail below.

The components of the molecular circuits of the present disclosure will generally be linked, functionally and/or physically, allowing a binding and/or activating event of one component to be transduced to another component of the circuit. For example, a nucleic acid encoding the antigen-specific therapeutic may be operably linked to a regulatory sequence and the regulatory sequence may be activated through the antigen-triggered transcriptional switch binding its cognate antigen.

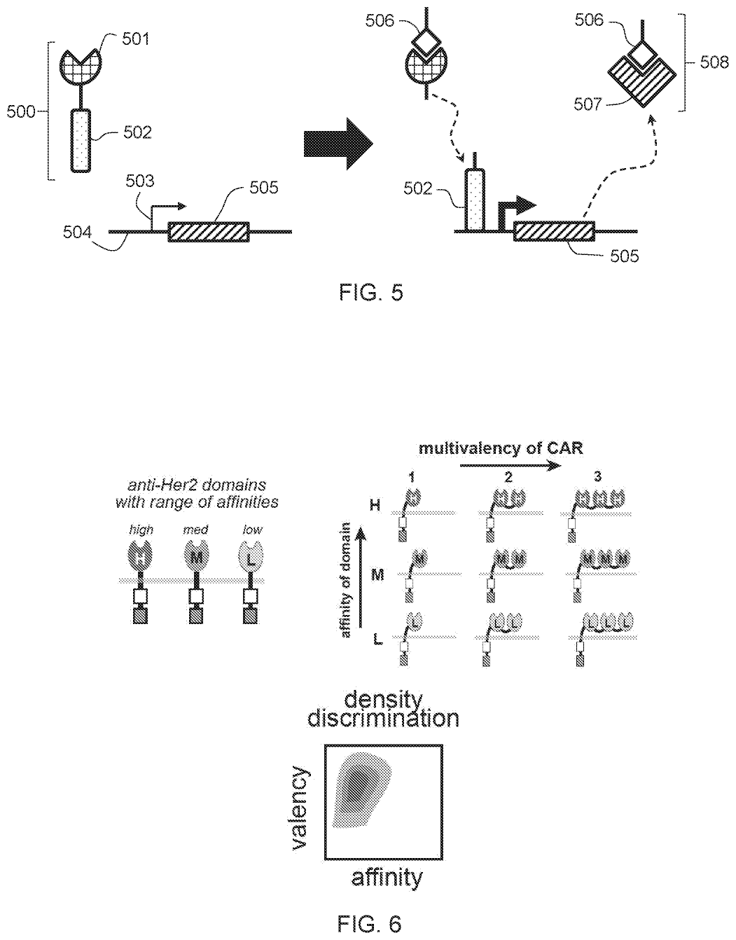

A schematic of this embodiment is depicted in FIG. 5. In the figure an antigen-triggered transcriptional switch 500 is shown with an antigen binding domain 501 and an intracellular domain 502 that, when released, activates a regulatory element 503, present on a nucleic acid 504, that drives expression of a sequence encoding an antigen-specific therapeutic 505. Upon binding the antigen 506, the intracellular domain 502 is released and therefore capable of driving expression of the sequence encoding the antigen-specific therapeutic 505, thereby producing the antigen-specific therapeutic 507. Once expressed, the antigen-specific therapeutic 507 can bind the antigen 506, which may initiate a therapeutic response mediated by the antigen-bound antigen specific therapeutic 508. In such a circuit, antigen-density sensing may be facilitated through differing affinities between the antigen and the antigen-triggered transcriptional switch compared to the antigen and the antigen-specific therapeutic, where e.g., the antigen-triggered transcriptional switch may have low affinity for the antigen and the antigen-specific therapeutic may have high affinity for the antigen.

### Affinity

Affinity, as it is used herein, may in some instances refer to the affinity of a specific binding domain, such as an antigen binding domain present on an antigen-triggered switch or an antigen-specific therapeutic. The affinity of a domain may be expressed in various context, including e.g., in isolation, when combined with or incorporated into a macromolecule, when present in a larger protein from which it is derived, etc. Accordingly, two domains may be said to have different affinities for a particular antigen and a first domain may be said to have a higher or lower affinity for the antigen relative to a second domain. In some instances, three or more domains may be ranked or ordered according to their affinity relative to one another, including e.g., where three domains are separately identified as high affinity, low affinity, and intermediate affinity.

In some instances, affinity may refer to an overall macromolecule, including e.g., where such a macromolecule has one or multiple antigen binding domains, rather than the affinity of an individual domain. For example, a macromolecule having multiple copies of an antigen binding domains could be expressed as having higher affinity than a similar macromolecule having only a single copy of the antigen binding domain.

As summarized above, circuits of the present disclosure will generally include an antigen-triggered transcriptional switch and an antigen-specific therapeutic that both bind to the same antigen but do so with differing affinity, including where the antigen-triggered transcriptional switch binds with low affinity to the antigen and the antigen-specific therapeutic binds with high affinity to the antigen. Accordingly, relevant affinities may be expressed in relative or absolute terms and may refer to an antigen binding domain present in an antigen-triggered transcriptional switch or an antigen-specific therapeutic or may refer to the entire an antigen-triggered transcriptional switch or an antigen-specific therapeutic, including where such macromolecules have multiple (e.g., 2, 3, 4, 5, 6, etc.) antigen binding domains.

In some instances, an antigen binding domain may bind to its cognate antigen or a macromolecule having one or multiple antigen binding domains may bind to one or multiple antigens with an affinity of at least 100 μM, including but not limited to e.g., at least 10 μM, at least 1 μM, at least 100 nM, at least 10 nM, or at least 1 nM.

In some instances, an antigen binding domain may bind to its cognate antigen or a macromolecule having one or multiple antigen binding domains may bind to one or multiple antigens with an affinity from about 10⁻⁴ M to about 5×10⁻⁴ M, from about 5×10⁻⁴ M to about 10⁻⁵ M, from about 10⁻⁵ M to 5×10⁻⁵ M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷ M, from about 5×10⁻⁷ M to about 10⁻⁸ M, from about 10⁻⁸ M to about 5×10⁻⁸ M, from about 5×10⁻⁸ M to about 10⁻⁹ M, from about 10⁻⁹ M to about 5×10⁻⁹, from about 5×10⁻⁹ M to about 10⁻¹⁰ M, from about 10⁻¹⁰ M to about 5×10⁻¹⁰, from about 5×10⁻¹⁰ M to about 10⁻¹¹ M, etc.

Expressed another way, in some instances, an antigen binding domain may bind to its cognate antigen or a macromolecule having one or multiple antigen binding domains may bind to one or multiple antigens with an affinity from about 0.01 nM to about 0.05 nM, from about 0.05 nM to about 0.1 nM, from about 0.1 nM to about 0.5 nM, from about 0.5 nM to about 1 nM, from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some instances, an antigen binding domain may bind to its cognate antigen or a macromolecule having one or multiple antigen binding domains may bind to one or multiple antigens with an affinity of at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the affinity of a corresponding antigen binding domain or macromolecule.

In some instances, an antigen binding domain may bind to its cognate antigen or a macromolecule having one or multiple antigen binding domains may bind to one or multiple antigens with an affinity of at least 10% more, at least 15% more, at least 20% more, at least 25% more, at least 30% more, at least 35% more, at least 40% more, at least 45% more, at least 50% more, at least 55% more, at least 60% more, at least 65% more, at least 70% more, at least 75% more, at least 80% more, at least 85% more, at least 90% more, at least 95% more, or more than 95% more than the affinity of a corresponding antigen binding domain or macromolecule.

In some instances, an antigen binding domain may bind to its cognate antigen or a macromolecule having one or multiple antigen binding domains may bind to one or multiple antigens with an affinity from 0.1 nM to 100 nM, or from 100 nM to 100 μM, including but not limited to e.g., from about 0.1 nM to 0.5 nM, from about 0.1 nM to 1 nM, from about 0.5 nM to 0.5 nM, from about 1 nM to 5 nM, from about 1 nM to 10 nM, from about 5 nM to 10 nM, from about 0.1 nM to 25 nM, from about 0.1 nM to 50 nM, from about 0.1 nM to 75 nM, from about 0.1 nM to 100 nM, from about 1 nM to 25 nM, from about 1 nM to 50 nM, from about 1 nM to 75 nM, from about 1 nM to 100 nM, from about 10 nM to 25 nM, from about 10 nM to 50 nM, from about 10 nM to 75 nM, from about 10 nM to 100 nM, from about 50 nM to 100 nM, from about 75 nM to 100 nM, from about 50 nM to 150 nM, from about 50 nM to 250 nM, from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 M to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

In some instances, an antigen binding domain may bind to its cognate antigen or a macromolecule having one or multiple antigen binding domains may bind to one or multiple antigens with an affinity that is at least 10%, at least 15%, at least 20%, at least 25%, at least 30%, at least 40%, at least 50%, at least 60%, at least 70%, at least 80%, at least 90%, at least 1.5-fold, at least 2-fold, at least 2.5-fold, at least 5-fold, at least 10-fold, at least 15-fold, at least 20-fold, at least 25-fold, at least 50-fold, at least 100-fold, at least 150-fold, at least 200-fold, at least 300-fold, at least 400-fold, at least 500-fold, at least 600-fold, at least 700-fold, at least 800-fold, at least 900-fold, at least 1000-fold, or more than 1000-fold, higher than a second antigen binding domain or macromolecule that binds the same antigen.

In some instances, an antigen binding domain may bind to its cognate antigen or a macromolecule having one or multiple antigen binding domains may bind to one or multiple antigens with an affinity that is at least 10%, at least 15%, at least 20%, at least 25%, at least 30%, at least 40%, at least 50%, at least 60%, at least 70%, at least 80%, at least 90%, at least 1.5-fold, at least 2-fold, at least 2.5-fold, at least 5-fold, at least 10-fold, at least 15-fold, at least 20-fold, at least 25-fold, at least 50-fold, at least 100-fold, at least 150-fold, at least 200-fold, at least 300-fold, at least 400-fold, at least 500-fold, at least 600-fold, at least 700-fold, at least 800-fold, at least 900-fold, at least 1000-fold, or more than 1000-fold, less than a second antigen binding domain or macromolecule that binds the same antigen.

In some instances, the difference in affinity for the antigen between two components of a subject circuit (e.g., a low affinity antigen-triggered transcriptional switch and a high affinity antigen specific therapeutic) may range from about at least 5-fold different to about at least 1000-fold different or more, including but not limited to e.g., from about at least 5-fold different to about at least 500-fold different, from about at least 5-fold different to about at least 250-fold different, from about at least 5-fold different to about at least 100-fold different, from about at least 5-fold different to about at least 50-fold different, from about at least 5-fold different to about at least 10-fold different, from about at least 10-fold different to about at least 1000-fold different, from about at least 20-fold different to about at least 1000-fold different, from about at least 50-fold different to about at least 1000-fold different, from about at least 100-fold different to about at least 1000-fold different, from about at least 250-fold different to about at least 1000-fold different, from about at least 500-fold different to about at least 1000-fold different, from about at least 10-fold different to about at least 500-fold different, from about at least 20-fold different to about at least 250-fold different, from about at least 100-fold different to about at least 500-fold different, from about at least 100-fold different to about at least 250-fold different, from about at least 10-fold different to about at least 100-fold different, or from about at least 100-fold different to about at least 250-fold different.

In the context of the herein described antigen-density sensing molecular circuits, what constitutes low affinity and high affinity for components of the circuit or the antigen binding domains thereof may vary. The difference between low and high affinity in a particular circuit need only sufficiently discriminate the antigen density threshold between targeted cells (e.g., cancer cells) and non-targeted cells (e.g., bystander cells). Accordingly, ranges of low affinity and high affinity may be adjusted based on the particular context of antigen expression by targeted cells and non-targeted cells. As such, as an example without limitation, in some instances a component of a herein described circuit with low affinity may have affinity for the antigen in the micromolar range (e.g., 1 to 10 μM) and a component of a herein described circuit with high affinity may have affinity for the antigen in the nanomolar range (e.g., 1 to 1000 nM). However, such ranges are not exclusive and may be altered based on the antigen density threshold needed to discriminate targeted cells from non-targeted cells, including e.g., where a low affinity component has an affinity for the antigen in the nanomolar range or where a high affinity component has an affinity for the antigen in the micromolar range. As used herein, high affinity and low affinity may reflect the relative affinities of two different components for the same antigen where the component with high affinity has a higher affinity for the antigen than the component with low affinity, including where the high affinity component and the low affinity component together have affinities sufficient to discriminate between target cells and non-target cells with antigen densities on either side of an antigen density threshold.

In some instances, the affinity of an antigen binding domain for an antigen, of the affinity of an antigen-binding macromolecule having one or multiple antigen binding domains, may be assessed, estimated, and/or quantitated in various ways. For example, in some instances, affinity may be assessed, estimated, and/or quantitated by a biochemical or biophysical method. Useful methods for assessing, estimating, and/or determining absolute and/or relative and/or estimated affinities may include but are not limited to e.g., affinity electrophoresis, bimolecular fluorescence complementation (BiFC), bio-layer interferometry, co-immunoprecipitation, dual polarisation interferometry (DPI), dynamic light scattering (DLS), flow-induced dispersion analysis (FIDA), fluorescence correlation spectroscopy, fluorescence polarization/anisotropy, fluorescence resonance energy transfer (FRET), isothermal titration calorimetry (ITC), microscale thermophoresis (MST), phage display, proximity ligation assay (PLA), quantitative immunoprecipitation combined with knock-down (QUICK), rotating cell-based ligand binding assay, static light scattering (SLS), single colour reflectometry (SCORE), surface plasmon resonance (SPR), tandem affinity purification (TAP), and the like.

The difference in measured affinity, regardless of the method of measurement employed, between two antigen binding domains, or antigen-binding macromolecules, may be expressed as a ratio such as but not limited to e.g., at least 1.5:1, at least 2:1, at least 5:1, at least 10:1, at least 15:1, at least 20:1, at least 25:1, at least 50:1, at least 100:1, at least 500:1, at least 102:1, at least 5×10²:1, at least 10′:1, at least 5×10′:1, at least 10⁴:1, at lease 10′:1, or at least 10⁶:1.

In some instances, an antigen binding domain may be modified, including where such modification increases or decreases the affinity of the antigen binding domain for its cognate antigen to generate affinity enhanced or affinity reduced versions of the subject antigen binding domain. Methods of generating antibody binding domains with enhanced affinity include but are not limited to e.g., in vitro affinity maturation, e.g., utilizing various display methods, as well as rational methods (see e.g., Rouet et al., Next-Generation Sequencing of Antibody Display Repertoires. Front Immunol. (2018) 9:118; Barderas et al., Affinity maturation of antibodies assisted by in silico modeling, Proc Natl Acad Sci USA. (2008) 105(26):9029-34; and Roskos L.; Klakamp S.; Liang M.; Arends R.; Green L. (2007). Stefan Dubel, ed. Handbook of Therapeutic Antibodies. Weinheim: Wiley-VCH. pp. 145-169; the disclosures of which are incorporated herein by reference in their entirety). Methods employed to generate antigen biding domains with enhanced affinity may also produce antigen binding domains with decreased affinity. In addition, when an antigen binding domain with enhanced affinity is produced, the parent (i.e., pre-modified) antigen binding domain may be employed as an antigen binding domain with lower affinity with respect to the enhanced version. Similarly, methods directed at humanizing non-human antibodies also regularly produce antigen binding domain variants with differing (including increased and decreased) affinities (see e.g., Carter et al., Proc Natl Acad Sci USA. (1992) 89:4285-89; the disclosure of which is incorporated herein by reference in its entirety). Nonetheless, methods of generating antibody binding domains with reduced affinity may also be employed, where such methods include but are not limited to e.g., random (untargeted) and targeted (directed) mutagenesis, alanine scanning, and screening (e.g., phage display, etc.) methods.

Antibodies, antigen binding domains and/or affinity enhanced and/or affinity reduced versions thereof, may be readily obtained from numerous commercial suppliers including but not limited to e.g., LakePharma (Belmont, Calif., USA), ModiQuest Research (Oss, Netherlands), Abzena (Babraham, United Kingdom), Oak Biosciences, Inc. (Sunnyvale, Calif., USA), Immune Corp. (Missoula, Mont., USA), Yurogen Biosystems LLC (Worcester, Mass., USA), Integral Molecular (Philadelphia, Pa., USA), AbBioSci (Seattle, Wash., USA), Rx Biosciences Ltd (Rockville, Md., USA), ImmunoPrecise Antibodies (Victoria, BC, Canada), AvantGen, Inc. (San Diego, Calif., USA), Revolve Biotechnologies, Inc. (Baltimore, Md., USA), Creative Biolabs (Shirley, N.Y., USA), and the like.

Antigen binding domains with desired affinity for an antigen may be incorporated into a component of the herein described circuits as desired. For example, an antigen binding domain with a low or reduced affinity may be incorporated into an antigen-triggered transcriptional switch, e.g., through recombination of nucleic acid sequences encoding the antigen binding domain and the antigen-triggered transcriptional switch. In some instances, an antigen binding domain with a high or increased affinity may be incorporated into an antigen specific therapeutic, e.g., through recombination of nucleic acid sequences encoding the antigen binding domain and the antigen specific therapeutic. Nucleic acid sequences encoding circuit components may be recombined with antigen binding domains of desired affinity with or without intervening sequences such as, e.g., linkers.

In some instances, the affinity of a macromolecule for an antigen may be modulated to achieve a desired affinity for the macromolecule for use in a circuit of the present disclosure. For example, the affinity of a macromolecule (e.g., an antigen-triggered transcriptional switch or an antigen specific therapeutic) for an antigen may be increased or decreased through the addition or removal of antigen binding domain(s) to or from the macromolecule, respectively. Accordingly, the valency of a macromolecule for an antigen may be increased or decreased, resulting in a corresponding increase of decrease in overall affinity of the macromolecule for the antigen.

An example of modifying the overall affinity of a macromolecule for an antigen by modulating the number of antigen binding domains is presented in FIG. 6. Using antigen-binding domains that bind the antigen Her2 with low, medium and high affinity as an example, FIG. 6 demonstrates that the affinity of the subject macromolecule may be increased by increasing the valency of the macromolecule for the antigen. Accordingly, the overall affinity of a subject multivalent macromolecule may be correspondingly decreased by decreasing the valency of the macromolecule, thus generating a macromolecule with decreased overall affinity for the antigen. As such, two macromolecules employed in a circuit of the present disclosure may have different affinity for an antigen based on having different valency. For example, an antigen-triggered transcriptional switch may have lower affinity for an antigen than an antigen specific therapeutic based on the antigen-triggered transcriptional switch having lower valency for the antigen than the antigen specific therapeutic. The valency of various macromolecules may vary and may include single valency of multivalency, including but not limited to e.g., 2, 3, 4, 5, 6 or more antigen binding domains present on a subject macromolecule.

### Antigens

Antigen-density sensing molecular circuits of the present disclosure may be configured to target essentially any desired antigen. Useful antigens to be targeted will generally include those antigens that are differentially expressed on target cells versus bystander cells, thus allowing targeting of the target cells and preventing targeting of the bystander cells based on differing antigen density between the cell types. Different available antigen binding domains having different affinities for an antigen may be selected for use in the herein described circuits. Alternatively, as described above, an antigen binding domain may be modified to produce a modified version with increased or decreased affinity, thus generating two antigen binding domains to the same antigen with differing affinity for the antigen for use in the herein described circuits. Accordingly, circuits of the present disclosure may be configured to target various different antigens and various different antigens may be targeted in methods employing the subject circuits as further described below.

Useful antigens that may be targeted using the circuits of the present disclosure include but are not limited to e.g., cancer antigens, i.e., an antigen expressed by (synthesized by) a neoplasia or cancer cell, i.e., a cancer cell associated antigen or a cancer (or tumor) specific antigen.

A cancer cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell, such as a bystander cell.

Non-limiting examples of cancer associated antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

A cancer cell specific antigen can be an antigen specific for cancer and/or a particular type of cancer or cancer cell including e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc.

A cancer (or tumor) specific antigen is generally not expressed by non-cancerous cells (or non-tumor cells). In some instances, a cancer (or tumor) specific antigen may be minimally expressed by one or more non-cancerous cell types (or non-tumor cell types). By “minimally expressed” is meant that the level of expression, in terms of either the per-cell expression level or the number of cells expressing, minimally, insignificantly or undetectably results in binding of antigen-binding domain containing macromolecules to non-cancerous cells expressing the antigen.

In some instances, a specific binding member may specifically bind a target comprising a fragment of a protein (e.g., a peptide) in conjunction with a major histocompatibility complex (MHC) molecule. As MHC molecules present peptide fragments of both intracellularly expressed and extracellularly expressed proteins, specific binding members directed to MHC-peptide complexes allows for the targeting of intracellular antigens as well as extracellularly expressed antigens. Peptides which may be targeted in the context of MHC include but are not limited to e.g., those described in PCT Pub. No. WO 2018/039247; the disclosure of which is incorporated herein by reference in its entirety.

Useful antigens also include surface expressed antigens. As used herein the term “surface expressed antigen” generally refers to antigenic proteins that are expressed at least partially extracellularly such that at least a portion of the protein is exposed outside the cell and available for binding with a binding partner. Essentially any surface expressed protein may find use as a target of an antigen-triggered transcriptional switch or antigen-specific therapeutic of the instant disclosure.

Non-limiting examples of useful antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. In some instances, useful antigens may be selected from: AFP, BCMA, CD10, CD117, CD123, CD133, CD138, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, Glypican 3, GPC3, HER-2, kappa immunoglobulin, LeY, LMP1, mesothelin, MG7, MUC1, NKG2D-ligands, PD-L1, PSCA, PSMA, ROR1, ROR1R, TACI and VEGFR2 and may include, e.g., an antigen binding-domain of or derived from a CAR currently or previously under investigation in one or more clinical trials.

In some instances, an antigen to which an antigen-density sensing circuit of the present disclosure is targeted is selected from Receptor tyrosine-protein kinase erbB-2 (HER2), CAMPATH-1 antigen (CD52), Programmed cell death 1 ligand 1 (PD-L1), Vascular endothelial growth factor (VEGF), B-lymphocyte antigen CD19 (CD19), Tumor necrosis factor receptor superfamily member 8 (CD30), Glutamate carboxypeptidase 2 (PSMA), Epidermal growth factor receptor (EGFR), disialoganglioside GD2 (GD2), SLAM family member 7 (SLAMF7), Myeloid cell surface antigen CD33 (CD33), B-lymphocyte antigen CD20 (CD20), B-cell receptor CD22 (CD22), Platelet-derived growth factor receptor alpha (PDGFRA), Vascular endothelial growth factor receptor 1 (VEGFR1), Vascular endothelial growth factor receptor 2 (VEGFR2), Mucin 1 (MCU1), Glutamate carboxypeptidase 2 (FOLH1), and Tyrosine-protein kinase receptor UFO (AXL). Accordingly, in some instances, a circuit of the present disclosure may include an antigen-triggered transcriptional switch and an antigen specific therapeutic that both target one of the foregoing antigens.

### Antigen Specific Therapeutics

As summarized above, the present circuits include an antigen-triggered transcriptional switch that, when bound to its cognate antigen, induces the expression of an antigen-specific therapeutic responsive to the antigen. Useful antigen-specific therapeutics will vary and may include surfaced expressed and secreted antigen-specific therapeutics. For example, in some instances, an antigen-specific therapeutic used in the methods of the present disclosure may be expressed, in response to the activation of an antigen-triggered transcriptional switch, on the surface of a cell, e.g., an immune cell, i.e., an immune cell genetically modified to encode a circuit as described herein. In some instances, an antigen-specific therapeutic used in the methods of the present disclosure may be secreted, in response to the activation of an antigen-triggered transcriptional switch, from a cell, e.g., an immune cell, i.e., an immune cell genetically modified to encode a circuit as described herein.

In general, except where described otherwise, the antigen-specific therapeutic of a herein described circuit will not be expressed in the absence of the activation of the antigen-triggered transcriptional switch that induces its expression. Also, except where described otherwise, an antigen-specific therapeutic of a herein described circuit will not be active in the absence of the antigen to which it binds, i.e., without binding the antigen to which the antigen-specific therapeutic is specific. Binding of its respective antigen, or antigens in the case of multi- or bispecific agents, results in activation of the antigen-specific therapeutic. When expressed by, or otherwise engaged with, an immune cell and bound to antigen(s) the antigen-specific therapeutic may activate the immune cell. Activated immune cells may mediate one or more beneficial effects with respect to a target cell, such as a cancer cell in a subject, including those beneficial effects described herein such as but not limited to e.g., cancer cell killing, cytokine release, and the like.

Antigen-specific therapeutics useful in the methods of the present disclosure will vary and may include but are not limited to e.g., chimeric antigen receptors (CARs), T cell receptors (TCRs), chimeric bispecific binding members, therapeutic antibodies, and the like.

Useful CARs include essentially any CAR useful in the treatment of cancer, including single-chain and multi-chain CARs, directed to the antigen to which the antigen-triggered transcriptional switch is targeted. A CAR used in the instant methods will generally include, at a minimum, an antigen binding domain, a transmembrane domain and an intracellular signaling domain. An employed CAR may further include one or more costimulatory domains.

Non-limiting examples of CARs that may be employed include those used in commercialized CAR T cell (CART) therapies including e.g., the anti-CD19-4-1BB—CD3ζ CAR expressed by lentivirus loaded CTL019 (Tisagenlecleucel-T) CAR-T cells, also referred to as Kymriah™ (tisagenlecleucel) as commercialized by Novartis (Basel, Switzerland), the anti-CD19-CD28-CD3ζ CAR of Yescart® (Axicabtagene Ciloleucel) commercialized by Kite Pharma, Inc. (Santa Monica, Calif.), and the anti-BCMA-4-1BB—CD3ζ CAR expressed by lentivirus loaded CAR-T cells called “bb2121” as investigated by bluebird bio, Inc. (Cambridge, Mass.) and Celgene Corporation (Summit, N.J.).

Useful CARs or useful domains thereof may, in some instances, include those described in U.S. Pat. Nos. 9,914,909; 9,821,012; 9,815,901; 9,777,061; 9,662,405; 9,657,105; 9,629,877; 9,624,276; 9,598,489; 9,587,020; 9,574,014; 9,573,988; 9,499,629; 9,446,105; 9,394,368; 9,328,156; 9,233,125; 9,175,308 and 8,822,647; the disclosures of which are incorporated herein by reference in their entirety. In some instances, useful CARs may include or exclude heterodimeric, also referred to as dimerizable or switchable, CARs and/or include or exclude one or more of the domains thereof. Useful heterodimeric CARs and/or useful domains thereof may, in some instances, include those described in U.S. Pat. Nos. 9,587,020 and 9,821,012 as well as U.S. Pub. Nos. US20170081411A1, US20160311901A1, US20160311907A1, US20150266973A1 and PCT Pub. Nos. WO2014127261A1, WO2015142661A1, WO2015090229A1 and WO2015017214A1; the disclosures of which are incorporated herein by reference in their entirety.

In some instances, the antigen binding domain of a CAR, such as but not limited to e.g., those described in any one of the documents referenced above, may be substituted or amended with an alternative or additional antigen binding domain directed to a different antigen, such as but not limited to one or more of the antigens described herein, for use in the herein described circuits. For example, in some instances, an antigen binding domain of a CAR may be substituted for an antigen binding domain having specificity for a different antigen. In some instances, an antigen binding domain of a CAR may be substituted for an antigen binding domain having higher or lower affinity for an antigen or such domain may be modified to have increased or decreased affinity for the antigen. In such instances, the intracellular portions (i.e., the intracellular signaling domain or the one or more co-stimulatory domains) of the antigen-domain-substituted CAR may or may not be modified.

In some embodiments, the antigen binding domain of a CAR may be or may be substituted for an antigen binding domain specific for an antigen selected from Receptor tyrosine-protein kinase erbB-2 (HER2), CAMPATH-1 antigen (CD52), Programmed cell death 1 ligand 1 (PD-L1), Vascular endothelial growth factor (VEGF), B-lymphocyte antigen CD19 (CD19), Tumor necrosis factor receptor superfamily member 8 (CD30), Glutamate carboxypeptidase 2 (PSMA), Epidermal growth factor receptor (EGFR), disialoganglioside GD2 (GD2), SLAM family member 7 (SLAMF7), Myeloid cell surface antigen CD33 (CD33), B-lymphocyte antigen CD20 (CD20), B-cell receptor CD22 (CD22), Platelet-derived growth factor receptor alpha (PDGFRA), Vascular endothelial growth factor receptor 1 (VEGFR1), Vascular endothelial growth factor receptor 2 (VEGFR2), Mucin 1 (MCU1), Glutamate carboxypeptidase 2 (FOLH1), and Tyrosine-protein kinase receptor UFO (AXL).

In some embodiments, the antigen binding domain of a CAR may be or may be derived from or may be a variant of an antibody useful in the treatment and/or diagnosis of cancer, such as but not limited to e.g., ado-trastuzumab emtansine (Kadcyla, Genentech) targeting HER2 as used in Metastatic breast cancer (the antibody described and/or referenced in U.S. Pat. Nos. 7,575,748 and 8,337,856); alemtuzumab (Campath, Lemtrada, Genzyme) targeting CD52 as used in B-cell chronic lymphocytic leukemia (the antibody described and/or referenced in U.S. Pat. Nos. 7,317,091 and 5,846,534); atezolizumab (Tecentriq, Genentech) targeting PD-L1 as used in Urothelial carcinoma and Metastatic non-small cell lung cancer (the antibody described and/or referenced in U.S. Pat. Nos. 9,873,740 and 8,217,149); avelumab (Bavencio, EMD Serono) targeting PD-L1 as used in Metastatic Merkel cell carcinoma (the antibody described and/or referenced in U.S. Pat. No. 9,676,863 and PCT Pub. WO2017097407); bevacizumab (Avastin, Genentech) targeting VEGF as used in Metastatic colorectal cancer, NSCLC, Glioblastoma, Metastatic renal cell carcinoma and cervical cancer (the antibody described and/or referenced in U.S. Pat. Nos. 7,575,893, 7,622,115 and 7,807,799); blinatumomab (Blincyto, Amgen) targeting CD19 as used in Precursor B-cell acute lymphoblastic leukemia (the antibody described and/or referenced in U.S. Pat. No. 8,076,459 and PCT Pub. WO2015006749); brentuximab vedotin (Adcentris, Seattle Genetics) targeting CD30 as used in Hodgkin lymphoma and Anaplastic large-cell lymphoma (the antibody described and/or referenced in U.S. Pat. No. 7,659,241); capromab pendetide (ProstaScint, Cytogen) targeting PSMA as used as a Diagnostic imaging agent in newly diagnosed prostate cancer or post-prostatectomy (the antibody described and/or referenced in U.S. Pat. Nos. 7,826,889, 8,420,081, 8,722,019, 8,883,146, 8,962,804, 9,211,315, and 9,364,567); cetuximab (Erbitux, ImClone Systems) targeting EGFR as used in Metastatic colorectal carcinoma (the antibody described and/or referenced in U.S. Pat. No. 9,120,853 and PCT Pub. WO2015000062); dinutuximab (Unituxin, United Therapeutics) targeting GD2 as used in Pediatric high-risk neuroblastoma (the antibody described and/or referenced in US Patent Applications US20160185841 and US20140170155); durvalumab (Imfinzi, AstraZeneca) targeting PD-L1 as used in Urothelial carcinoma (the antibody described and/or referenced in U.S. Pat. No. 8,779,108 and PCT Pub. WO 2018068201); elotuzumab (Empliciti, Bristol-Myers Squibb) targeting SLAMF7 as used in Multiple myeloma (the antibody described and/or referenced in US Patent Application US20170002060 and PCT Pub. WO2014055370); gemtuzumab ozogamicin (Mylotarg, Wyeth) targeting CD33 as used in Acute myeloid leukemia (the antibody described and/or referenced in U.S. Pat. Nos. 5,693,761 and 7,727,968); ibritumomab tiuxetan (Zevalin, Spectrum Pharmaceuticals) targeting CD20 as used in Relapsed or refractory low-grade, follicular, or transformed B-cell non-Hodgkin's lymphoma (the antibody described and/or referenced in U.S. Pat. Nos. 5,736,137, 5,776,456, 5,843,439, 6,207,858, 6,399,061, 6,682,734, 6,994,840, 7,229,620 and 8,906,681); inotuzumab ozogamicin (Besponsa, Wyeth) targeting CD22 as used in Precursor B-cell acute lymphoblastic leukemia (the antibody described and/or referenced in U.S. patent application Ser. No. 10/428,894); ipilimumab (Yervoy, Bristol-Myers Squibb) targeting CTLA-4 as used in Metastatic melanoma (the antibody described and/or referenced in U.S. Pat. Nos. 8,993,524 and 7,605,238); necitumumab (Portrazza, Eli Lilly) targeting EGFR as used in Metastatic squamous non-small cell lung carcinoma (the antibody described and/or referenced in U.S. Pat. Nos. 8,962,804 and 7,598,350); nivolumab (Opdivo, Bristol-Myers Squibb) targeting PD-1 as used in Metastatic melanoma and Metastatic squamous non-small cell lung carcinoma (the antibody described and/or referenced in U.S. Pat. No. 9,724,413); obinutuzumab (Gazyva, Genentech) targeting CD20 as used in Chronic lymphocytic leukemia (the antibody described and/or referenced in U.S. Pat. Nos. 6,602,684, 7,517,670, and 8,021,856); ofatumumab (Arzerra, Glaxo Grp) targeting CD20 as used in Chronic lymphocytic leukemia (the antibody described and/or referenced in U.S. Pat. No. 9,949,971 and PCT Pub. WO2004035607); olaratumab (Lartruvo, Eli Lilly) targeting PDGFRA as used in Soft tissue sarcoma (the antibody described and/or referenced in U.S. Pat. Nos. 8,128,929 and 8,574,578); panitumumab (Vectibix, Amgen) targeting EGFR as used in Metastatic colorectal cancer (the antibody described and/or referenced in U.S. Pat. No. 6,235,883); pembrolizumab (Keytruda, Merck) targeting PD-1 as used in Metastatic melanoma (the antibody described and/or referenced in U.S. Pat. No. 9,827,309 amd 8,952,136); pertuzumab (Perjeta, Genentech) targeting HER2 as used in Metastatic breast cancer (the antibody described and/or referenced in U.S. Pat. No. 9,513,296); ramucirumab (Cyramza, Eli Lilly) targeting VEGFR2 as used in Gastric cancer (the antibody described and/or referenced in US Patent Application US20170002060 and PCT Pub. WO2003075840); rituximab (Rituxan, Genentech) targeting CD20 as used in B-cell non-Hodgkin's lymphoma (the antibody described and/or referenced in U.S. Pat. No. 8,815,242 and European Patent Nos. EP0605442 and EP0669836); rituximab and hyaluronidase (Rituxan Hycela, Genentech) targeting CD20 as used in Follicular lymphoma, Diffuse large B-cell lymphoma and Chronic lymphocytic leukemia (the antibody described and/or referenced in European Patent No. EP2475353); trastuzumab (Herceptin, Genentech) targeting HER2 as used in Metastatic breast cancer, HER2-overexpressing breast cancer, metaststic gastric or gastroesophageal junction adenocarcinoma (the antibody described and/or referenced in U.S. Pat. Nos. 9,753,040, 6,407,213 and 6,331,415); and the like.

In some instances, a CAR useful in the herein described circuits may be an affinity tuned CAR, such as but not limited to e.g., an affinity tuned Her2 (ErbB2) CAR or an affinity tuned EGFR CAR, including but not limited to e.g., one or more of the CARs described in Liu et al., (2015) Cancer Res 75(17):3596-3607; the disclosure of which is incorporated herein by reference in its entirety.

Useful CARs and/or useful domains thereof may, in some instances, include those that have been or are currently being investigated in one or more clinical trials, including but not limited to the CARs directed to the following antigens (listed with an exemplary corresponding clinical trial number, further information pertaining to which may be retrieved by visiting www(dot)clinicaltrials(dot)gov): AFP, e.g., in NCT03349255; BCMA, e.g., in NCT03288493; CD10, e.g., in NCT03291444; CD117, e.g., in NCT03291444; CD123, e.g., in NCT03114670; CD133, e.g., in NCT02541370; CD138, e.g., in NCT01886976; CD171, e.g., in NCT02311621; CD19, e.g., in NCT02813252; CD20, e.g., in NCT03277729; CD22, e.g., in NCT03244306; CD30, e.g., in NCT02917083; CD33, e.g., in NCT03126864; CD34, e.g., in NCT03291444; CD38, e.g., in NCT03291444; CD5, e.g., in NCT03081910; CD56, e.g., in NCT03291444; CD7, e.g., in NCT02742727; CD70, e.g., in NCT02830724; CD80, e.g., in NCT03356808; CD86, e.g., in NCT03356808; CEA, e.g., in NCT02850536; CLD18, e.g., in NCT03159819; CLL-1, e.g., in NCT03312205; cMet, e.g., in NCT01837602; EGFR, e.g., in NCT03182816; EGFRvIII, e.g., in NCT02664363; EpCAM, e.g., in NCT03013712; EphA2, e.g., in NCT02575261; GD-2, e.g., in NCT01822652; Glypican 3, e.g., in NCT02905188; GPC3, e.g., in NCT02723942; HER-2, e.g., in NCT02547961; kappa immunoglobulin, e.g., in NCT00881920; LeY, e.g., in NCT02958384; LMP1, e.g., in NCT02980315; mesothelin, e.g., in NCT02930993; MG7, e.g., in NCT02862704; MUC1, e.g., in NCT02587689; NKG2D-ligands, e.g., in NCT02203825; PD-L1, e.g., in NCT03330834; PSCA, e.g., in NCT02744287; PSMA, e.g., in NCT03356795; ROR1, e.g., in NCT02706392; ROR1R, e.g., in NCT02194374; TACI, e.g., in NCT03287804; and VEGFR2, e.g., in NCT01218867.

In some instances, the antigen binding domain of a previously investigated CAR, such but not limited to e.g., tisagenlecleucel or bb2121 or a CAR that has been or is currently being investigated in a clinical trial as listed above, may be substituted or amended with an alternative or additional antigen binding domain directed to a different antigen, such as but not limited to one or more of the antigens described herein, for use in the herein described methods. In such instances, the intracellular portions (i.e., the intracellular signaling domain or the one or more co-stimulatory domains) of the antigen-domain-substituted CAR may or may not be modified.

Useful TCRs include essentially any TCR useful in the treatment of cancer, including single-chain and multi-chain TCRs, directed to a targeting antigen. A TCR used in the instant methods will generally include, at a minimum, an antigen binding domain and a modified or unmodified TCR chain, or portion thereof, including but not limited to e.g., a modified or unmodified α-chain, a modified or unmodified β-chain, etc. An employed TCR may further include one or more costimulatory domains. In some instances, a TCR employed herein will include an alpha chain and a beta chain and recognize antigen when presented by a major histocompatibility complex.

Essentially any TCR can be induced by an antigen-triggered transcriptional switch using a method of the present disclosure including e.g., TCRs that are specific for any of a variety of epitopes, including, e.g., an epitope expressed on the surface of a cancer cell, a peptide-MHC complex on the surface of cancer cell, and the like. In some cases, the TCR is an engineered TCR.

Non-limiting examples of engineered TCRs, including those having immune cell activation function, useful in the methods described herein include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710⁻²¹; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety.

In some instances, a circuit of the described methods involves the induction of an engineered TCR targeting a cancer antigen. In some instances, an engineered TCR induced to be expressed in a circuit of the instant disclosure is an engineered TCR targeting an antigen target listed in the following table.

Engineered TCR Targets:

| Target | HLA | References |
|---|---|---|
| NY-ESO-1 | HLA-A2 | J Immunol. (2008) 180(9):6116-31 |
| MART-1 | HLA A2 | J Immunol. (2008) 180(9):6116-31; |
| | | Blood. (2009) 114(3):535-46 |
| MAGE-A3 | HLA-A2 | J Immunother. (2013) 36(2):133-51 |
| MAGE-A3 | HLA-A1 | Blood. (2013) 122(6):863-71 |
| CEA | HLA-A2 | Mol Ther. (2011) 19(3):620-626 |
| gp100 | HLA-A2 | Blood. (2009) 114(3):535-46 |
| WT1 | HLA-A2 | Blood. (2011) 118(6):1495-503 |
| HBV | HLA-A2 | J Hepatol. (2011) 55(1):103-10 |
| gag (WT | HLA-A2 | Nat Med. (2008) 14(12):1390-5 |
| and/or | | |
| α/6) | | |
| P53 | HLA-A2 | Hum Gene Ther. (2008) |
| | | 19(11):1219-32 |
| TRAIL | N/A | J Immunol. (2008) 181(6):3769-76 |
| bound | | |
| to DR4 | | |
| HPV-16 | HLA-A2 | Clin Cancer Res. (2015) |
| (E6 and/or | | 21(19):4431-9 |
| E7) | | |
| Survivin | HLA-A2 | J Clin Invest. (2015) 125(1):157-68 |
| KRAS | HLA-A11 | Cancer Immunol Res. (2016) |
| mutants | | 4(3):204-14 |
| SSX2 | HLA-A2 | PLoS One. (2014) 9(3):e93321 |
| MAGE- | HLA-A2 | J ImmunoTherapy Cancer. (2015) |
| A10 | | 3(Suppl2):P14 |
| MAGE-A4 | HLA-A24 | Clin Cancer Res. (2015) |
| | | 21(10):2268-77 |
| AFP | HLA-A2 | J ImmunoTherapy Cancer. (2013) |
| | | 1(Suppl1):P10 |

In some instances, an expressed TCR targeting a particular antigen may be described as an anti-[antigen] TCR. Accordingly, in some instances, exemplary TCRs that may be induced to be expressed in the methods of the instant disclosure include but are not limited to e.g., an anti-NY-ESO-1 TCR; an anti-MART-1 TCR; an anti-MAGE-A3 TCR; an anti-MAGE-A3 TCR; an anti-CEA TCR; an anti-gp100 TCR; an anti-WT1 TCR; an anti-HBV TCR; an anti-gag (WT and/or a/6) TCR; an anti-P53 TCR; an anti-TRAIL bound to DR4 TCR; an anti-HPV-16 (E6 and/or E7) TCR; an anti-Survivin TCR; an anti-KRAS mutants TCR; an anti-SSX2 TCR; an anti-MAGE-A10 TCR; an anti-MAGE-A4 TCR; an anti-AFP TCR; and the like.

Useful TCRs include those having wild-type affinity for their respective antigen as well as those having enhanced affinity for their respective antigen. TCRs having enhanced affinity for their respective antigen may be referred to as “affinity enhanced” or “enhanced affinity” TCRs. The affinity of a TCR may be enhanced by any convenient means, including but not limited to binding-site engineering (i.e., rational design), screening (e.g., TCR display), or the like. Non-limiting examples of affinity enhanced TCRs and methods of generating enhanced affinity TCRs include but are not limited to e.g., those described in PCT Pub. Nos. 20150118208, 2013256159, 20160083449; 20140349855, 20100113300, 20140371085, 20060127377, 20080292549, 20160280756, 20140065111, 20130058908, 20110038842, 20110014169, 2003276403 and the like; the disclosures of which are incorporated herein by reference in their entirety. Further engineered TCRs, modifications thereof, that may be expressed in response to release of an intracellular domain of an antigen-triggered transcriptional switch of the present disclosure include e.g., those described in PCT Application No. US2017/048040; the disclosure of which is incorporated herein by reference in its entirety.

Useful TCRs may, in some instances, also include those described in U.S. Pat. Nos. 9,889,161; 9,889,160; 9,868,765; 9,862,755; 9,717,758; 9,676,867; 9,409,969; 9,115,372; 8,951,510; 8,906,383; 8,889,141; 8,722,048; 8,697,854; 8,603,810; 8,383,401; 8,361,794; 8,283,446; 8,143,376; 8,003,770; 7,998,926; 7,666,604; 7,456,263; 7,446,191; 7,446,179; 7,329,731; 7,265,209; and 6,770,749; the disclosures of which are incorporated herein by reference in their entirety.

In some instances, the antigen binding domain of a TCR, such as but not limited to e.g., those described or referenced above, may be substituted or amended with an alternative or additional antigen binding domain directed to a different antigen, such as but not limited to one or more of the antigens described herein, for use in the herein described methods. In such instances, the other portions (i.e., the transmembrane domain, any intracellular signaling domains, etc.) of the antigen-domain-substituted TCR may or may not be modified.

As summarized above, in some instances, useful antigen-specific therapeutics may include those that, upon induction by an activated antigen-triggered transcriptional switch, are expressed and secreted from the producing cell, including e.g., where the secreting cell is an immune cell. For example, upon binding of an antigen-triggered transcriptional switch expressed by an immune cell, the antigen-triggered transcriptional switch may induce expression and secretion of an encoded antigen-specific therapeutic specific for the antigen.

Useful secreted antigen-specific therapeutics will vary and, in some instances, may include but are not limited to e.g., chimeric bispecific binding members, antibodies, and the like.

Useful antibodies include those antibodies that are useful in or have been investigated for the therapeutic treatment of cancer. Non-limiting examples of therapeutic antibodies for the treatment of cancer include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MED14736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

In some instances, useful antibodies, or the antigen binding domains thereof, may also include 8H9, Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBSO7, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

In some instances, useful chimeric bispecific binding members may include those that target a protein expressed on the surface of an immune cell, including but not limited to e.g., a component of the T cell receptor (TCR), e.g., one or more T cell co-receptors. Chimeric bispecific binding members that bind to a component of the TCR may be referred to herein as a TCR-targeted bispecific binding agent. Chimeric bispecific binding members useful in the instant methods will generally be specific for a targeting antigen and may, in some instances, be specific for a targeting antigen and a protein expressed on the surface of an immune cell (e.g., a component of a TCR such as e.g., a CD3 co-receptor).

In some instances, useful chimeric bispecific binding members may include a bispecific T cell engager (BiTE). A BiTE is generally made by fusing a specific binding member (e.g., a scFv) that binds an immune cell antigen to a specific binding member (e.g., a scFv) that binds a cancer antigen (e.g., a tumor associated antigen, a tumor specific antigen, etc.). For example, an exemplary BiTE includes an anti-CD3 scFv fused to an anti-tumor associated antigen (e.g., EpCAM, CD19, etc.) scFv via a short peptide linker (e.g., a five amino acid linker, e.g., GGGGS). In some instances, a BiTE suitable for use as herein described methods may include e.g., an anti-CD3×anti-CD19 BiTE (e.g., Blinatumomab), an anti-EpCAM×anti-CD3 BiTE (e.g., MT110), an anti-CEA×anti-CD3 BiTE (e.g., MT111/MEDI-565), an anti-CD33×anti-CD3 BiTE, an anti-HER2 BiTE, an anti-EGFR BiTE, an anti-IgE BiTE, and the like.

In some instances, the antigen binding domain of a chimeric bispecific binding member, such as but not limited to e.g., those described or referenced above, may be substituted or amended with an alternative or additional antigen binding domain directed to a different antigen, such as but not limited to one or more of the antigens described herein, for use in the herein described methods. In such instances, the other portions (i.e., linker domain, any immune cell targeting domains, etc.) of the antigen-domain-substituted chimeric bispecific binding member may or may not be modified.

As summarized above, antigen binding domains of antigen specific therapeutics may be substituted, amended or exchanged as desired. For example, an antigen binding domain of an antibody described above may be employed as the antigen binding domain of a CAR. Correspondingly, an antigen binding domain described above as used in a CAR may be employed in other contexts, such as e.g., in an antibody or a chimeric bispecific binding member or the like. As such, disclosure above of any agent targeted to a specific antigen in the context of a particular antigen specific therapeutic would be understood to constitute a disclosure of the use of an antigen binding domain for the antigen in any other antigen specific therapeutic in the herein described circuits as well.

### Antigen-Triggered Transcriptional Switches

As summarized above, the present circuits include the production of an antigen-specific therapeutic driven by a regulatory sequence that is induced by the activation of an antigen-triggered transcriptional switch, where such activation is caused by the antigen-triggered transcriptional switch binding its cognate antigen. Useful antigen-triggered transcriptional switches in the herein described circuits will vary.

As used herein, a “antigen-triggered transcriptional switch” generally refers to a synthetic modular polypeptide or system of interacting polypeptides having an extracellular domain that includes a first member of a specific binding pair that binds an antigen (i.e., the second member of the specific binding pair), a binding-transducer and an intracellular domain. Upon binding of the second member of the specific binding pair to the antigen-triggered transcriptional switch the binding signal is transduced to the intracellular domain such that the intracellular domain becomes activated and performs some function within the cell that it does not perform in the absence of the binding signal. Certain antigen triggered transcriptional switches, as also in some instances referred to as binding-triggered transcriptional switches, are described in e.g., PCT Pub. No. WO 2016/138034 as well as U.S. Pat. Nos. 9,670,281 and 9,834,608; the disclosures of which are incorporated herein by reference in their entirety.

The specific binding member of the extracellular domain generally determines the specificity of the antigen-triggered transcriptional switch. In some instances, an antigen-triggered transcriptional switch may be referred according to its specificity as determined based on its specific binding member. For example, a specific binding member having binding partner “X” may be referred to as an X-antigen-triggered transcriptional switch or an anti-X antigen-triggered transcriptional switch.

Any convenient and appropriate specific binding pair, i.e., specific binding member and specific binding partner pair, may find use in the antigen-triggered transcriptional switch of the instant methods including but not limited to e.g., antigen-antibody pairs, ligand receptor pairs, scaffold protein pairs, etc.

In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a scaffold protein and its binding partner may be a protein to which the scaffold protein specifically binds.

Useful specific binding pairs include those specific for an antigen, including those antigens described herein. For simplicity, regardless of the actual nature of the binding pair (i.e., antigen/antibody, receptor/ligand, etc.), the member of the binding pair attached to the antigen triggered transcriptional switch will be referred to herein as an antigen binding domain and the member to which it binds will be referred to as an antigen herein (i.e., regardless of whether such a molecule would otherwise be considered an “antigen” in the conventional sense). However, one of ordinary skill will readily understand that descriptions of antigen binding domain-antigen interactions can be substituted with ligand/receptor, scaffold/binding partner pair where desired as appropriate.

In some cases, the specific binding member is an antibody. The antibody can be any antigen-binding antibody-based polypeptide, a wide variety of which are known in the art. In some instances, the specific binding member is or includes a monoclonal antibody, a single chain Fv (scFv), a Fab, etc. Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

Where the specific binding member is an antibody-based binding member, the antigen-triggered transcriptional switch can be activated in the presence of a binding partner to the antibody-based binding member, including e.g., an antigen specifically bound by the antibody-based binding member. In some instances, antibody-based binding member may be defined, as is commonly done in the relevant art, based on the antigen bound by the antibody-based binding member, including e.g., where the antibody-based binding member is described as an “anti-” antigen antibody, e.g., an anti-CD19 antibody. Accordingly, antibody-based binding members suitable for inclusion in an antigen-triggered transcriptional switch or an antigen-specific therapeutic of the present methods can have a variety of antigen-binding specificities.

The components of antigen-triggered transcriptional switches, employed in the described methods, and the arrangement of the components of the switch relative to one another will vary depending on many factors including but not limited to e.g., the desired antigen, the activity of the intracellular domain, the overall function of the antigen-triggered transcriptional switch, the broader arrangement of a molecular circuit comprising the antigen-triggered transcriptional switch, etc. The first binding member may include but is not limited to e.g., those agents that bind an antigen described herein. The intracellular domain may include but is not limited e.g., those intracellular domains that activate or repress transcription at a regulatory sequence, e.g., to induce or inhibit expression of a downstream component of a particular circuit.

The binding transducer of antigen-triggered transcriptional switches will also vary depending on the desired method of transduction of the binding signal. Generally, binding transducers may include those polypeptides and/or domains of polypeptides that transduce an extracellular signal to intracellular signaling e.g., as performed by the receptors of various signal transduction pathways. Transduction of a binding signal may be achieved through various mechanisms including but not limited to e.g., binding-induced proteolytic cleavage, binding-induced phosphorylation, binding-induced conformational change, etc. In some instances, a binding-transducer may contain a ligand-inducible proteolytic cleavage site such that upon binding the binding-signal is transduced by cleavage of the antigen-triggered transcriptional switch, e.g., to liberate an intracellular domain. For example, in some instances, an antigen-triggered transcriptional switch may include a Notch derived cleavable binding transducer, such as, e.g., a chimeric notch receptor polypeptide as described herein.

In other instances, the binding signal may be transduced in the absence of inducible proteolytic cleavage. Any signal transduction component or components of a signaling transduction pathway may find use in an antigen-triggered transcriptional switch whether or not proteolytic cleavage is necessary for signal propagation. For example, in some instances, a phosphorylation-based binding transducer, including but not limited to e.g., one or more signal transduction components of the Jak-Stat pathway, may find use in a non-proteolytic antigen-triggered transcriptional switch.

For simplicity, antigen-triggered transcriptional switches, including but not limited to chimeric notch receptor polypeptides, are described primarily as single polypeptide chains. However, antigen-triggered transcriptional switches, including chimeric notch receptor polypeptides, may be divided or split across two or more separate polypeptide chains where the joining of the two or more polypeptide chains to form a functional antigen-triggered transcriptional switch, e.g., a chimeric notch receptor polypeptide, may be constitutive or conditionally controlled. For example, constitutive joining of two portions of a split antigen-triggered transcriptional switch may be achieved by inserting a constitutive heterodimerization domain between the first and second portions of the split polypeptide such that upon heterodimerization the split portions are functionally joined.

Useful antigen-triggered transcriptional switches that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful antigen-triggered transcriptional switches that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful antigen-triggered transcriptional switches that may be employed in the subject methods include, but are not limited to von Willebrand Factor (vWF) cleavage domain-based BTTS's, such as but not limited to e.g., those containing an unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTS's are described in Langridge & Struhl (Cell (2017) 171(6):1383-1396); the disclosure of which is incorporated herein by reference in its entirety.

Useful antigen-triggered transcriptional switches that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety.

SynNotch polypeptides are generally proteolytically cleavable chimeric polypeptides that generally include: a) an extracellular domain comprising a specific binding member; b) a proteolytically cleavable Notch receptor polypeptide comprising one or more proteolytic cleavage sites; and c) an intracellular domain. Binding of the specific binding member by its binding partner generally induces cleavage of the synNotch at the one or more proteolytic cleavage sites, thereby releasing the intracellular domain. In some instances, the instant methods may include where release of the intracellular domain triggers (i.e., induces) the production of an encoded payload, the encoding nucleic acid sequence of which is contained within the cell. Depending on the particular context, the produced payload is then generally expressed on the cell surface or secreted. SynNotch polypeptides generally include at least one sequence that is heterologous to the Notch receptor polypeptide (i.e., is not derived from a Notch receptor), including e.g., where the extracellular domain is heterologous, where the intracellular domain is heterologous, where both the extracellular domain and the intracellular domain are heterologous to the Notch receptor, etc.

Useful synNotch antigen-triggered transcriptional switches will vary in the domains employed and the architecture of such domains. SynNotch polypeptides will generally include a Notch receptor polypeptide that includes one or more ligand-inducible proteolytic cleavage sites. The length of Notch receptor polypeptides will vary and may range in length from about 50 amino acids or less to about 1000 amino acids or more.

In some cases, the Notch receptor polypeptide present in a synNotch polypeptide has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a synNotch polypeptide has a length of from 300 aa to 400 aa, from 300 aa to 350 aa, from 300 aa to 325 aa, from 350 aa to 400 aa, from 750 aa to 850 aa, from 50 aa to 75 aa. In some cases, the Notch receptor polypeptide has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide has a length of 315 aa. In some cases, the Notch receptor polypeptide has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide has a length of 367 aa.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence.

Subject Notch regulatory regions may include or exclude various components (e.g., domains, cleavage sites, etc.) thereof. Examples of such components of Notch regulatory regions that may be present or absent in whole or in part, as appropriate, include e.g., one or more EGF-like repeat domains, one or more Lin12/Notch repeat domains, one or more heterodimerization domains (e.g., HD-N or HD-C), a transmembrane domain, one or more proteolytic cleavage sites (e.g., a furin-like protease site (e.g., an S1 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta-binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

Non-limiting examples of particular synNotch antigen-triggered transcriptional switches, the domains thereof, and suitable domain arrangements are described in PCT Pub. Nos. WO 2016/138034, WO 2017/193059, WO 2018/039247 and U.S. Pat. Nos. 9,670,281 and 9,834,608; the disclosures of which are incorporated herein by reference in their entirety.

Domains of a useful antigen-triggered transcriptional switch, e.g., the extracellular domain, the binding-transducer domain, the intracellular domain, etc., may be joined directly, i.e., with no intervening amino acid residues or may include a peptide linker that joins two domains. Peptide linkers may be synthetic or naturally derived including e.g., a fragment of a naturally occurring polypeptide.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

In some instances, an antigen-triggered transcriptional switch may have an extracellular domain that includes a first member of a specific binding pair that binds a second member of the specific binding pair, wherein the extracellular domain does not include any additional first or second member of a second specific binding pair. For example, in some instances, an antigen-triggered transcriptional switch may have an extracellular domain that includes a first antigen-binding domain that binds an antigen, wherein the extracellular domain does not include any additional antigen-binding domains and does not bind any other antigens. A subject antigen-triggered transcriptional switch may, in some instances, include only a single extracellular domain. Accordingly, an employed antigen-triggered transcriptional switch may be specific for a single antigen and only specific for the single antigen. Such, antigen-triggered transcriptional switches may be referred to as a “single antigen antigen-triggered transcriptional switch”.

Antigen-triggered transcriptional switches specific for a single antigen may be monovalent or multivalent (e.g., bivalent, trivalent, etc.) for the antigen. For example, in some instances, a monovalent antigen-triggered transcriptional switch may be employed that includes an antigen binding domain (e.g., a single antigen binding domain) for binding a single molecule of antigen. In some instances, a multivalent antigen-triggered transcriptional switch may be employed that includes an antigen binding domain or multiple antigen binding domains (e.g., 1, 2, 3, 4, 5, 6, etc. antigen binding domains) for binding multiple molecules of antigen.

In some instances, an antigen-triggered transcriptional switch may have an extracellular domain that includes the first or second members of two or more specific binding pairs. For example, in some instances, an antigen-triggered transcriptional switch may have an extracellular domain that includes a first antigen-binding domain and a second antigen-binding domain that are different such that the extracellular domain is specific for two different antigens. In some instances, an antigen-triggered transcriptional switch may have two or more extracellular domains that each includes the first or second members of two different specific binding pairs. For example, in some instances, an antigen-triggered transcriptional switch may have a first extracellular domain that includes a first antigen-binding domain and a second extracellular domain that includes a second antigen-binding domain where the two different antigen binding domains are each specific for a different antigen. As such, the antigen-triggered transcriptional switch may be specific for two different antigens.

An antigen-triggered transcriptional switch specific for two or more different antigens, containing either two extracellular domains or one extracellular domain specific for two different antigens, may be configured such that the binding of either antigen to the antigen-triggered transcriptional switch is sufficient to trigger activation of the antigen-triggered transcriptional switch, e.g., proteolytic cleavage of a cleavage domain of the antigen-triggered transcriptional switch, e.g., releasing an intracellular domain of the antigen-triggered transcriptional switch. Such an antigen-triggered transcriptional switch, capable of being triggered by any of two or more antigens, may find use in the described circuits as a component of a logic gate containing OR functionality. In some instances, an antigen-triggered transcriptional switch specific for two different antigens may be referred to as a “two-headed antigen-triggered transcriptional switch”. Antigen-triggered transcriptional switches specific for multiple antigens will not be limited to only two antigens and may, e.g., be specific for and/or triggered by more than two antigens, including e.g., three or more, four or more, five or more, etc.

As summarized above, antigen binding domains of antigen-triggered transcriptional switches may be substituted, amended or exchanged as desired. For example, an antigen binding domain of an antigen specific therapeutic, such as an antibody described above, may be employed as the antigen binding domain of an antigen-triggered transcriptional switch described herein. Correspondingly, an antigen binding domain described above as used in a CAR may be employed in other contexts, such as e.g., in an antigen-triggered transcriptional switch as described above. As such, disclosure above of any agent targeted to a specific antigen in any context herein would be understood to constitute a disclosure of the use of an antigen binding domain for the antigen in any antigen-triggered transcriptional switch in the herein described circuits as well.

### Nucleic Acids

As summarized above, the present disclosure also provides nucleic acids encoding the molecular circuits described herein, including but not limited to where such nucleic acids are included in expression constructs, vectors, cells and the like.

The subject nucleic acids may include, e.g., a sequence encoding an antigen-triggered transcriptional switch and a sequence encoding an antigen-specific therapeutic. Such nucleic acids may be configured such that the sequence encoding the antigen-specific therapeutic is operably linked to a regulatory sequence responsive to activation of the antigen-triggered transcriptional switch. Provided are nucleic acids encoding essentially any circuit utilizing an antigen-density sensing molecular circuit, including but not limited to those circuits specifically described herein. Encompassed are isolated nucleic acids encoding the subject circuits as well as various configurations containing such nucleic acids, such as vectors, e.g., expression cassettes, recombinant expression vectors, viral vectors, and the like.

Recombinant expression vectors of the present disclosure include those comprising one or more of the described nucleic acids. A nucleic acid comprising a nucleotide sequence encoding all or a portion of the components of a circuit of the present disclosure will in some embodiments be DNA, including, e.g., a recombinant expression vector. A nucleic acid comprising a nucleotide sequence encoding all or a portion of the components of a circuit of the present disclosure will in some embodiments be RNA, e.g., in vitro synthesized RNA.

As summarized above, in some instances, the subject circuits may make use of an encoding nucleic acid (e.g., a nucleic acid encoding an antigen-triggered transcriptional switch or an antigen-specific therapeutic) that is operably linked to a regulatory sequence such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is an immune cell promoter such as a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-0 gene promoter, a WASP gene promoter, a T-cell receptor β-chain gene promoter, a V9 γ (TRGV9) gene promoter, a V2 δ (TRDV2) gene promoter, and the like.

In some cases, a nucleic acid comprising a nucleotide sequence encoding a circuit of the present disclosure, or one or more components thereof, is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a circuit of the present disclosure, or one or more components thereof, is a recombinant lentivirus vector.

In some cases, a nucleic acid comprising a nucleotide sequence encoding a circuit of the present disclosure, or one or more components thereof, is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements, may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence.

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag.

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21):1411-1423; Martin et al. (2006) BMC Biotechnology. 6:4; the disclosures of which are incorporated herein by reference in their entirety.

### Cells

As summarized above, the present disclosure also provides cells encoding the molecular circuits described herein, including nucleic acids described herein encoding such molecular circuits. Such cells may be genetically modified with the herein described nucleic acids by a variety of means, including but not limited to where nucleic acids are introduced as or using expression constructs, vectors (viral or non-viral), transfection (non-viral, such as electroporation, lipofection, biolistics, etc.), and the like. Any polypeptide of interest may be encoded from a nucleic acid within a cell operably linked to a transcription control element responsive to an antigen-triggered transcriptional switch in the circuits of the present disclosure.

The activity of essentially any suitable antigen specific therapeutic can be controlled in an antigen-density dependent manner in any eukaryotic cell employing a circuit of the present disclosure. In some cases, the cell is in vivo. In some cases, the cell is ex vivo. In some cases, the cell is in vitro. In some cases, the cell is a mammalian cell. In some cases, the cell is a human cell. In some cases, the cell is a non-human primate cell. In some cases, the cell is rodent cell. In some cases, the cell is mouse cell. In some cases, the cell is a rat cell.

Suitable cells may vary and may include, in some instances, immune cells. Immune cells of the present disclosure include mammalian immune cells including e.g., those that are genetically modified to produce the components of a circuit of the present disclosure or to which a nucleic acid, as described above, has been otherwise introduced. In some instances, the subject immune cells have been transduced or transfected with one or more nucleic acids and/or expression vectors to express one or more components of a circuit of the present disclosure.

Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a T lymphocyte, a stem cell, or an NK cell is obtained from an individual; and the cell obtained from the individual is modified to express components of a circuit of the present disclosure. The modified cell can thus be redirected to one or more antigens of choice, as defined by the one or more antigen binding domains present on the introduced components of the circuit. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

In some cases, the cell is genetically modified to express two different heterologous or non-endogenous polypeptides of a herein described circuit. In some instances, the cell is genetically modified to express two or more different heterologous or non-endogenous polypeptides of a herein described circuit, including two or more different heterologous or non-endogenous polypeptides of the present disclosure, including but not limited to e.g., 2 different heterologous or non-endogenous polypeptides of the present disclosure, 3 different heterologous or non-endogenous polypeptides of the present disclosure, 4 different heterologous or non-endogenous polypeptides of the present disclosure, 5 different heterologous or non-endogenous polypeptides of the present disclosure, etc.

### Methods

As summarized above, the present disclosure also provides methods of making antigen-density sensing molecular circuits, methods of inducing expression of high affinity therapeutics specific to an antigen expressed by a target cell, methods of activating an immune response to a target cell, methods of treating a subject for a cancer expressing an antigen, and the like, where such methods involve antigen-density sensing molecular circuits.

Methods of the present disclosure include methods of modifying a cellular behavior, such as e.g., causing a cell to express a desired component from a nucleic acid sequence encoding the component in particular cellular contexts. For example, in some instances, an antigen-density sensing circuit of the present disclosure may be employed to induce expression of a high affinity therapeutic that is specific for an antigen expressed by a target cell, such as a cancer cell. Such circuits may be configured to induce activation of the cell (e.g., immune cell) containing the circuit when the cell is in the presence of a target cell (e.g., cancer cell) expressing the relevant antigen at a high level. Such circuits may be configured to induce activation of the cell (e.g., immune cell) containing the circuit when the cell is in the presence of a target cell (e.g., cancer cell) expressing the relevant antigen above an antigen-density threshold determined by the relative affinities of the components of the circuit (e.g., the relative affinities to the antigen of the antigen-triggered transcriptional switch and the antigen specific therapeutic).

Such circuits may be configured to prevent expression of the high affinity therapeutic by a cell containing the circuit, when the cell is not in the presence of a target cell expressing the relevant antigen at high density. Such circuits may also be configured to prevent expression of the high affinity therapeutic by a cell containing the circuit, when the cell is in the presence of a non-target cell (e.g., a bystander cell) that expresses the relevant antigen at low density. In some instances, such circuits may also be configured to prevent expression of the high affinity therapeutic by a cell containing the circuit, when the cell is in the presence of a non-target cell (e.g., a bystander cell) that expresses the relevant antigen at a density that is below an antigen-density threshold.

Such circuits may be configured to prevent or be insufficient to induce activation of the cell (e.g., immune cell) containing the circuit when the cell is in the presence of a non-target cell (e.g., a bystander cell) expressing the relevant antigen at a low level. In some instances, Such circuits may be configured to prevent or be insufficient to induce activation of the cell (e.g., immune cell) containing the circuit when the cell is in the presence of a non-target cell (e.g., a bystander cell) expressing the relevant antigen below an antigen-density threshold determined by the relative affinities of the components of the circuit (e.g., the relative affinities to the antigen of the antigen-triggered transcriptional switch and the antigen specific therapeutic).

Such methods may include administering to a subject a cell genetically modified to include a molecular circuit that includes an antigen-triggered transcriptional switch that binds with low affinity to an antigen of the targeted cell, wherein binding of the antigen-triggered transcriptional switch to the antigen induces expression of the high affinity therapeutic in the subject.

The methods of the present disclosure may involve activating an immune response to a target cell (e.g., a cancer cell) through the use of an immune cell that contains an antigen-density sensing circuit of the present disclosure. For example, methods of the present disclosure may, in some instances, include administering to a subject an immune cell genetically modified to include a molecular circuit that includes an antigen-triggered transcriptional switch that binds with low affinity to an antigen of the targeted cell to induce expression of an antigen-specific therapeutic that binds with high affinity to the antigen to activate the immune response in the subject. In some instances, inducing an immune response to a target cell will effectively treat the subject for a condition caused by target cell, such as cancer.

### Methods of Treating

As summarized above, provided are methods of treating a subject for disorder caused by a target cell, such as a cancer where the target cell is a cancerous cell such as a tumor cell of a solid cancer or a cell of a liquid cancer, such as a blood cancer.

Desired effects of the treatments, as described in more detail below, will vary. For example, with respect to the various cell types present in the subject, desired effects may include but are not limited to e.g., killing one or more targeted cell types, reducing the proliferation of the one or more targeted cell types, and the like. The method of the present disclosure may further include not affecting or minimal affecting a non-targeted cell type that also expressed the targeted antigen, including but not limited to e.g., not killing or minimally killing one or more non-targeted cell types, not reducing or minimally reducing the proliferation of one or more non-targeted cell types. By “minimally affecting”, e.g., “minimally killing”, “minimally reducing the proliferation of”, etc., is generally meant that the effect on the non-targeted cell type is at least less than would be expected if the instant methods of antigen-density sensing were not employed and may include where the subject experiences fewer, or does not experience any, side effects or adverse events as a result of off-targeting of non-targeted cells.

The subject methods may include introducing into a subject in need thereof, cells that contain nucleic acid sequences encoding a circuit for antigen-density dependent targeting of a cancer cell. In some instances, the subject may be known to contain bystander cells that express the antigen used to target the cancer. In some instances, the presence of bystander cells may be unknown. The introduced cells may be immune cells, including e.g., myeloid cells or lymphoid cells.

In some instances, the instant methods may include contacting a cell with one or more nucleic acids encoding a circuit wherein such contacting is sufficient to introduce the nucleic acid(s) into the cell. Any convenient method of introducing nucleic acids into a cell may find use herein including but not limited viral transfection, electroporation, lipofection, bombardment, chemical transformation, use of a transducible carrier (e.g., a transducible carrier protein), and the like. Nucleic acids may be introduced into cells maintained or cultured in vitro or ex vivo. Nucleic acids may also be introduced into a cell in a living subject in vivo, e.g., through the use of one or more vectors (e.g., viral vectors) that deliver the nucleic acids into the cell without the need to isolate, culture or maintain the cells outside of the subject.

Introduced nucleic acids may be maintained within the cell or transiently present. As such, in some instance, an introduced nucleic acid may be maintained within the cell, e.g., integrated into the genome. Any convenient method of nucleic acid integration may find use in the subject methods, including but not limited to e.g., viral-based integration, transposon-based integration, homologous recombination-based integration, and the like. In some instance, an introduced nucleic acid may be transiently present, e.g., extrachromosomally present within the cell. Transiently present nucleic acids may persist, e.g., as part of any convenient transiently transfected vector.

An introduced nucleic acid encoding a circuit may be introduced in such a manner as to be operably linked to a regulatory sequence, such as a promoter, that drives the expression of one or more components of the circuit. The source of such regulatory sequences may vary and may include e.g., where the regulatory sequence is introduced with the nucleic acid, e.g., as part of an expression construct or where the regulatory sequence is present in the cell prior to introducing the nucleic acid or introduced after the nucleic acid. As described in more detail herein, useful regulatory sequence can include e.g., endogenous promoters and heterologous promoters. For example, in some instances, a nucleic acid may be introduced as part of an expression construct containing a heterologous promoter operably linked to a nucleic acid sequence. In some instances, a nucleic acid may be introduced as part of an expression construct containing a copy of a promoter that is endogenous to the cell into which the nucleic acid is introduced. In some instances, a nucleic acid may be introduced without a regulatory sequence and, upon integration into the genome of the cell, the nucleic acid may be operably linked to an endogenous regulatory sequence already present in the cell. Depending on the confirmation and/or the regulatory sequence utilized, expression of each component of the circuit from the nucleic acid may be configured to be constitutive, inducible, tissue-specific, cell-type specific, etc., including combinations thereof.

Any convenient method of delivering the circuit encoding components may find use in the subject methods. In some instances, the subject circuit may be delivered by administering to the subject a cell expressing the circuit. In some instances, the subject circuit may be delivered by administering to the subject a nucleic acid comprising one or more nucleotide sequences encoding the circuit. Administering to a subject a nucleic acid encoding the circuit may include administering to the subject a cell containing the nucleic acid where the nucleic acid may or may not yet be expressed. In some instances, administering to a subject a nucleic acid encoding the circuit may include administering to the subject a vector designed to deliver the nucleic acid to a cell.

Accordingly, in the subject methods of treatment, nucleic acids encoding a circuit or components thereof may be administered in vitro, ex vivo or in vivo. In some instances, cells may be collected from a subject and transfected with nucleic acid and the transfected cells may be administered to the subject, with or without further manipulation including but not limited to e.g., in vitro expansion. In some instances, the nucleic acid, e.g., with or without a delivery vector, may be administered directly to the subject.

As summarized above, the methods described herein may be employed to treat a subject having a cancer, including where the subject also has bystander cells that express an antigen used to target the cancer. In some instances, the cancer is a tumor, such as a solid tumor. Cancer cells of a cancer targeted in the methods of the present disclosure may be in the proximity of a bystander cells expressing an antigen used to target the cancer. In some instances, bystander cells expressing the targeted antigen may be distant from the cancer.

In some instances, a targeted cell expresses an antigen more highly (i.e., more densely) than the antigen is expressed by one or more bystander cell types. The difference in level may vary but will generally be sufficiently different to allow for antigen-density discrimination according to the circuits and methods described herein. In some instances, the difference in antigen expression may be less than one order of magnitude. In some instances, the difference in antigen expression may be one order of magnitude or more, including but not limited to e.g., from less than one order of magnitude of expression to ten orders of magnitude of expression or more, including but not limited to e.g., 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 orders of magnitude, 10 orders of magnitude, etc.

The methods of the present disclosure may be employed to target and treat a variety of cancers, including e.g., primary cancer, secondary cancers, re-growing cancers, recurrent cancers, refractory cancers and the like. For example, in some instances, the methods of the present disclosure may be employed as an initial treatment of a primary cancer identified in a subject. In some instances, the methods of the present disclosure may be employed as a non-primary (e.g., secondary or later) treatment, e.g., in a subject with a cancer that is refractory to a prior treatment, in a subject with a cancer that is re-growing following a prior treatment, in a subject with a mixed response to a prior treatment (e.g., a positive response to at least one tumor in the subject and a negative or neutral response to at least a second tumor in the subject), and the like.

The instant methods may be employed for the treatment of various cancers including but not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sözary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy.

With respect to the cancer as a whole, desired effects of the described treatments may result in a reduction in the number of cells in the cancer, a reduction in the size of a tumor, a reduction in the overall proliferation of the cancer, a reduction in the overall growth rate of a tumor, etc. For example, an effective treatment is in some cases a treatment that, when administered in one or more doses to an individual in need thereof, reduces the number of cancer cells in the individual and/or reduces tumor mass in the individual, by at least about 10%, at least about 15%, at least about 20%, at least about 25%, at least about 30%, at least about 40%, at least about 50%, at least about 75%, or more than 75%, compared to the number of cancer cells and/or tumor mass in the absence of the treatment.

In some embodiments, an effective treatment is a treatment that, when administered alone (e.g., in monotherapy) or in combination (e.g., in combination therapy) with one or more additional therapeutic agents, in one or more doses, is effective to reduce one or more of tumor growth rate, cancer cell number, and tumor mass, by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the tumor growth rate, cancer cell number, or tumor mass in the absence of the treatment.

In some instances, treatment may involve activation of an immune cell containing nucleic acid sequences encoding a circuit as described herein. Accordingly, the present disclosure correspondingly presents methods of activating an immune cell, e.g., where the immune cell expresses an antigen-density sensing circuit as described herein.

Immune cell activation, as a result of the methods described herein, may be measured in a variety of ways, including but not limited to e.g., measuring the expression level of one or more markers of immune cell activation. Useful markers of immune cell activation include but are not limited to e.g., CD25, CD38, CD40L (CD154), CD69, CD71, CD95, HLA-DR, CD137 and the like. For example, in some instances, upon antigen binding by an immune cell receptor an immune cell may become activated and may express a marker of immune cell activation (e.g., CD69) at an elevated level (e.g., a level higher than a corresponding cell not bound to antigen). Levels of elevated expression of activated immune cells of the present disclosure will vary and may include an increase, such as a 1-fold or greater increase in marker expression as compared to un-activated control, including but not limited to e.g., a 1-fold increase, a 2-fold increase, a 3-fold increase, a 4-fold increase, etc.

In some instances, an immune cell modified to encode a circuit of the present disclosure, when bound to a targeted antigen, may have increased cytotoxic activity, e.g., as compared to an un-activated control cell. In some instances, activated immune cells encoding a subject circuit may show 10% or greater cell killing of antigen expressing target cells as compared to un-activated control cells. In some instances, the level of elevated cell killing of activated immune cells will vary and may range from 10% or greater, including but not limited to e.g., 20% or greater, 30% or greater, 40% or greater, 50% or greater, 60% or greater, 70% or greater, 80% or greater, 90% or greater, etc., as compared to an appropriate control.

In some instances, treatment may involve modulation, including induction, of the expression and/or secretion of a cytokine by an immune cell containing nucleic acid sequences encoding a circuit as described herein. Non-limiting examples of cytokines, the expression/secretion of which may be modulated, include but are not limited to e.g., Interleukins and related (e.g., IL-1-like, IL-1α, IL-1β, IL-1RA, IL-18, IL-2, IL-4, IL-7, IL-9, IL-13, IL-15, IL-3, IL-5, GM-CSF, IL-6-like, IL-6, IL-11, G-CSF, IL-12, LIF, OSM, IL-10-like, IL-10, IL-20, IL-14, IL-16, IL-17, etc.), Interferons (e.g., IFN-α, IFN-0, IFN-γ, etc.), TNF family (e.g., CD154, LT-β, TNF-α, TNF-β, 4-1BBL, APRIL, CD70, CD153, CD178, GITRL, LIGHT, OX40L, TALL-1, TRAIL, TWEAK, TRANCE, etc.), TGF-β family (e.g., TGF-β1, TGF-β2, TGF-β3, etc.) and the like.

In some instances, activation of an immune cell through a circuit of the present disclosure may induce an increase in cytokine expression and/or secretion relative to that of a comparable cell where the circuit is not present or otherwise inactive. The amount of the increase may vary and may range from a 10% or greater increase, including but not limited to e.g., 10% or greater, 25% or greater, 50% or greater, 75% or greater, 100% or greater, 150% or greater, 200% or greater, 250% or greater, 300% or greater, 350% or greater 400% or greater, etc.

### Methods of Making

The present disclosure further includes methods of making the nucleic acids, circuits, and cells, including those employed in the herein described methods. In making the subject nucleic acids and circuits, and components thereof, any convenient methods of nucleic acid manipulation, modification and amplification (e.g., collectively referred to as “cloning”) may be employed. In making the subject cells, containing the nucleic acids encoding the described circuits, convenient methods of transfection, transduction, culture, etc., may be employed.

A nucleotide sequence encoding all or a portion of the components of a circuit of the present disclosure can be present in an expression vector and/or a cloning vector. Where a subject circuit or component thereof is split between two or more separate polypeptides, nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRITS (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins.

A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a circuit or component thereof of the present disclosure will in some embodiments be DNA or RNA, e.g., in vitro synthesized DNA, recombinant DNA, in vitro synthesized RNA, recombinant RNA, etc. Methods for in vitro synthesis of DNA/RNA are known in the art; any known method can be used to synthesize DNA/RNA comprising a desired sequence. Methods for introducing DNA/RNA into a host cell are known in the art. Introducing DNA/RNA into a host cell can be carried out in vitro or ex vivo or in vivo. For example, a host cell (e.g., an NK cell, a cytotoxic T lymphocyte, etc.) can be transduced, transfected or electroporated in vitro or ex vivo with DNA/RNA comprising a nucleotide sequence encoding all or a portion of a circuit of the present disclosure.

Methods of the instant disclosure may further include culturing a cell genetically modified to encode a circuit of the instant disclosure including but not limited to e.g., culturing the cell prior to administration, culturing the cell in vitro or ex vivo (e.g., the presence or absence of one or more antigens), etc. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

Methods of the instant disclosure may, in some instances, further include receiving and/or collecting cells that are used in the subject methods. In some instances, cells are collected from a subject. Collecting cells from a subject may include obtaining a tissue sample from the subject and enriching, isolating and/or propagating the cells from the tissue sample. Isolation and/or enrichment of cells may be performed using any convenient method including e.g., isolation/enrichment by culture (e.g., adherent culture, suspension culture, etc.), cell sorting (e.g., FACS, microfluidics, etc.), and the like. Cells may be collected from any convenient cellular tissue sample including but not limited to e.g., blood (including e.g., peripheral blood, cord blood, etc.), bone marrow, a biopsy, a skin sample, a cheek swab, etc. In some instances, cells are received from a source including e.g., a blood bank, tissue bank, etc. Received cells may have been previously isolated or may be received as part of a tissue sample thus isolation/enrichment may be performed after receiving the cells and prior to use. In certain instances, received cells may be non-primary cells including e.g., cells of a cultured cell line. Suitable cells for use in the herein described methods are further detailed herein.

Methods of making nucleic acids, circuits and/or cells of the present disclosure may also include generating a modified antigen binding domain, e.g., an antigen binding domain with modified affinity for its antigen, including increased or decreased. Methods of generating modified antigen binding domains with reduced or increased affinity may vary and may include but are not limited to e.g., those described herein such as e.g., in vitro affinity maturation, rational design, random (untargeted) and targeted (directed) mutagenesis, alanine scanning, and affinity screening (e.g., phage display, etc.), and related methods.

Methods of making nucleic acids, circuits and/or cells of the present disclosure may also include obtaining, isolating, copying, cloning, recombining, duplicating, amplifying, and/or sequencing one or more antigen binding domains. For example, in some instances, an obtained antigen binding domain may be recombined into a component of a circuit (e.g., an antigen specific therapeutic) as described herein and a modified (e.g., mutated) form of the antigen binding domain (e.g., with reduced affinity for its antigen) may be recombined into a second component of the circuit (e.g., an antigen-triggered transcriptional switch) as described herein.

Methods of making nucleic acids, circuits and/or cells of the present disclosure may include essentially any combination of molecular biological procedures as desired to produce a nucleic acid, circuit or cell as described herein. For example, in some instances, an appropriate and desired set of molecular biological procedures may be employed to generate a molecular circuit encoding an antigen-triggered transcriptional switch comprising an antigen binding domain that, when activated, induces expression of an antigen-specific therapeutic comprising a higher affinity modified antigen binding domain. In some instances, an appropriate and desired set of molecular biological procedures may be employed to generate a molecular circuit encoding an antigen-triggered transcriptional switch comprising a lower affinity modified antigen binding domain that, when activated, induces expression of an antigen-specific therapeutic comprising the antigen binding domain.

### Kits

The present disclosure provides a kit for carrying out a method as described herein and/or constructing one or more circuits, components thereof, nucleic acids encoding a circuit or a component thereof, etc. In some cases, a subject kit comprises a vector, e.g., an expression vector or a delivery vector, comprising a nucleotide sequence encoding a circuit of the present disclosure or one or more portions thereof. Delivery vectors may be provided in a delivery device or may be provided separately, e.g., as a kit that includes the delivery vector and the delivery device as separate components of the kit.

In some cases, a subject kit comprises a cell, e.g., a host cell or host cell line, that is or is to be genetically modified with a nucleic acid comprising nucleotide sequence encoding a circuit of the present disclosure or a portion thereof. In some cases, a subject kit comprises a cell, e.g., a host cell, that is or is to be genetically modified with a recombinant expression vector comprising a nucleotide sequence encoding a circuit of the present disclosure. Kit components can be in the same container, or in separate containers.

Any of the above-described kits can further include one or more additional reagents, where such additional reagents can be selected from: a dilution buffer; a reconstitution solution; a wash buffer; a control reagent; a control expression vector; a nucleic acid encoding a negative control (e.g., a circuit that lacks the one or more critical elements); a nucleic acid encoding a positive control polypeptide; and the like.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. An antigen-density sensing molecular circuit comprising:
   (a) a nucleic acid sequence encoding an antigen-triggered transcriptional switch that binds with low affinity to an antigen present on the surface of a target cell;
   (b) a nucleic acid sequence encoding an antigen-specific therapeutic that binds with high affinity to the antigen; and
   (c) a regulatory sequence operably linked to (b) that is activated by binding of the antigen-triggered transcriptional switch to the antigen to induce expression of the antigen-specific therapeutic.
2. The molecular circuit according to aspect 1, wherein the target cell is a cancer cell.
3. The molecular circuit according to aspect 2, wherein the antigen is selected from the group consisting of: Receptor tyrosine-protein kinase erbB-2 (HER2), CAMPATH-1 antigen (CD52), Programmed cell death 1 ligand 1 (PD-L1), Vascular endothelial growth factor (VEGF), B-lymphocyte antigen CD19 (CD19), Tumor necrosis factor receptor superfamily member 8 (CD30), Glutamate carboxypeptidase 2 (PSMA), Epidermal growth factor receptor (EGFR), disialoganglioside GD2 (GD2), SLAM family member 7 (SLAMF7), Myeloid cell surface antigen CD33 (CD33), B-lymphocyte antigen CD20 (CD20), B-cell receptor CD22 (CD22), Platelet-derived growth factor receptor alpha (PDGFRA), Vascular endothelial growth factor receptor 1 (VEGFR1), Vascular endothelial growth factor receptor 2 (VEGFR2), Mucin 1 (MCU1), Glutamate carboxypeptidase 2 (FOLH1), and Tyrosine-protein kinase receptor UFO (AXL).
4. The molecular circuit according to any of the preceding aspects, wherein the antigen-specific therapeutic comprises a single antigen-binding domain specific for the antigen.
5. The molecular circuit according to any of aspects 1 to 3, wherein the antigen-specific therapeutic comprises multiple antigen-binding domains specific for the antigen.
6. The molecular circuit according to any of the preceding aspects, wherein the antigen-triggered transcriptional switch comprises a single antigen-binding domain specific for the antigen.
7. The molecular circuit according to any of aspects 1 to 5, wherein the antigen-triggered transcriptional switch comprises multiple antigen-binding domains specific for the antigen.
8. The molecular circuit according to any of the preceding aspects, wherein the antigen-specific therapeutic is a chimeric antigen receptor (CAR), a T cell receptor (TCR), or an antibody.
9. The molecular circuit according to any of the preceding aspects, wherein the antigen-triggered transcriptional switch comprises a Notch force sensor cleavage domain.
10. The molecular circuit according to aspect 9, wherein the antigen-triggered transcriptional switch is a synNotch polypeptide.
11. The molecular circuit according to any of aspects 1 to 8, wherein the antigen-triggered transcriptional switch comprises a non-Notch force sensor cleavage domain.
12. The molecular circuit according to aspect 11, wherein the non-Notch force sensor cleavage domain comprises a von Willebrand Factor (vWF) cleavage domain.
13. A cell genetically modified to comprise the molecular circuit of any of the preceding aspects.
14. The cell of aspect 13, wherein the cell is an immune cell.
15. The cell of aspect 14, wherein the immune cell is a myeloid cell or a lymphoid cell.
16. The cell of aspect 15, wherein the immune cell is a lymphoid cell selected from the group consisting of: a T lymphocyte, a B lymphocyte and a Natural Killer cell.
17. The cell of any of aspects 13 to 16, wherein the antigen-specific therapeutic is expressed on the surface of the cell.
18. The cell of any of aspects 13 to 16, wherein the antigen-specific therapeutic is secreted by the cell.
19. A method of making an antigen-density sensing molecular circuit, the method comprising:
obtaining a sequence encoding an antigen binding domain that binds to an antigen;
generating a modified antigen binding domain sequence encoding:
   a high affinity modified antigen binding domain with increased affinity for the antigen as compared to the antigen binding domain; or
   a low affinity modified antigen binding domain with decreased affinity for the antigen as compared to the antigen binding domain; and
generating a molecular circuit encoding an antigen-triggered transcriptional switch comprising the antigen binding domain that, when activated, induces expression of an antigen-specific therapeutic comprising the high affinity modified antigen binding domain; or
generating a molecular circuit encoding an antigen-triggered transcriptional switch comprising the low affinity modified antigen binding domain that, when activated, induces expression of an antigen-specific therapeutic comprising the antigen binding domain.
20. The method according to aspect 19, wherein the antigen-specific therapeutic is a chimeric antigen receptor (CAR), a T cell receptor (TCR), or an antibody.
21. The method according to aspects 19 or 20, wherein the antigen-triggered transcriptional switch comprises a Notch force sensor cleavage domain.
22. The method according to aspect 21, wherein the antigen-triggered transcriptional switch is a synNotch polypeptide.
23. The method according to aspects 19 or 20, wherein the antigen-triggered transcriptional switch comprises a non-Notch force sensor cleavage domain.
24. The method according to aspect 23, wherein the non-Notch force sensor cleavage domain comprises a von Willebrand Factor (vWF) cleavage domain.
25. A method of inducing expression of a high affinity therapeutic specific to an
antigen expressed by a target cell in a subject in need thereof, the method comprising:
administering to the subject a cell genetically modified to comprise a molecular circuit comprising an antigen-triggered transcriptional switch that binds with low affinity to the antigen, wherein binding of the antigen-triggered transcriptional switch to the antigen induces expression of the high affinity therapeutic in the subject.
26. The method according to aspect 25, wherein the antigen is a cancer antigen and the target cell is a cancer cell.
27. The method according to aspects 25 or 26, wherein the high affinity therapeutic is a chimeric antigen receptor (CAR), a T cell receptor (TCR), or an antibody.
28. The method according to any of aspects 25-27, wherein the genetically modified cell is a cell according to any one of aspects 13 to 18.
29. A method of activating an immune response to a target cell expressing an antigen in a subject; the method comprising:
administering to the subject an immune cell genetically modified to comprise a molecular circuit comprising an antigen-triggered transcriptional switch that binds with low affinity to the antigen to induce expression of an antigen-specific therapeutic that binds with high affinity to the antigen to activate the immune response in the subject.
30. The method according to aspect 29, wherein the molecular circuit comprises an antigen-density sensing molecular circuit according to any one of aspects 1 to 12.
31. A method of treating a subject for a cancer expressing an antigen, the method comprising:
administering to the subject an effective amount of immune cells genetically modified to comprise a molecular circuit comprising an antigen-triggered transcriptional switch that binds with low affinity to the antigen to induce expression of an antigen-specific therapeutic that binds with high affinity to the antigen to activate an immune response in the subject, thereby treating the subject for the cancer.
32. The method according to aspect 31, wherein the antigen is also expressed by non-cancer cells in the subject.
33. The method according to aspects 31 or 32, wherein the effective amount of immune cells comprise an immune cell according to any one of aspects 14 to 16.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Her2 Density Sensing synNotch/CAR Circuit Selectively Kills High Density Her2 Cancer Cells

Current strategies for generating CAR T cells consist of selecting antibodies with high affinity because previous studies have shown that the CAR T cell activity is inversely correlated with antibody affinity. However, these conventional CARs are unable to discriminate between cancer and normal cells, where such cells differ in antigen density. Therapeutic T cells can ideally distinguish clearly high antigen density expressing tumor cells from normal cells that express lower levels of a tumor associated antigen. A CAR T cell with a standard linear response curve would distinguish poorly between high and low density cells, while good discrimination requires a sigmoidal ultrasensitive dose-response curve (FIG. 1A). Common mechanisms for ultrasensitive sensing: molecular ultrasensitivity can be achieved by a multivalent cooperative sensor (e.g. hemoglobin), but cellular circuit ultrasensitivity can be achieved through multi-step sensing with positive feedback (e.g. IL-2 sensing by the IL-2 receptor induces a second step—expression of CD25, a high affinity IL-2 receptor subunit) (FIG. 1B).

Lowering the receptor affinity on a CAR T cell could increase its selectivity against target cells with different antigen densities. However, lowering receptor affinity is unlikely to provide a sharp density threshold of activation, which would allow for the discrimination between high antigen density expressing cancer cells and low antigen density expressing bystander cells.

For a sharp threshold transition based on antigen density, cooperativity and non-linear recognition is desirable. For this purpose, a two-step recognition-activation circuit that involves two receptors has been designed. In this circuit an initial recognition event alters the potency of a subsequent response. In particular, a weak receptor is employed that turns on the activity of a high affinity receptor that fully activates the cell. In this example, the subject circuits make use of modular receptors called synthetic notch receptors (SynNotch). SynNotch receptors use antibody-based domains to recognize a target antigen, and when activated, the receptor controls transcription via a transcriptional domain released by a cleavage event.

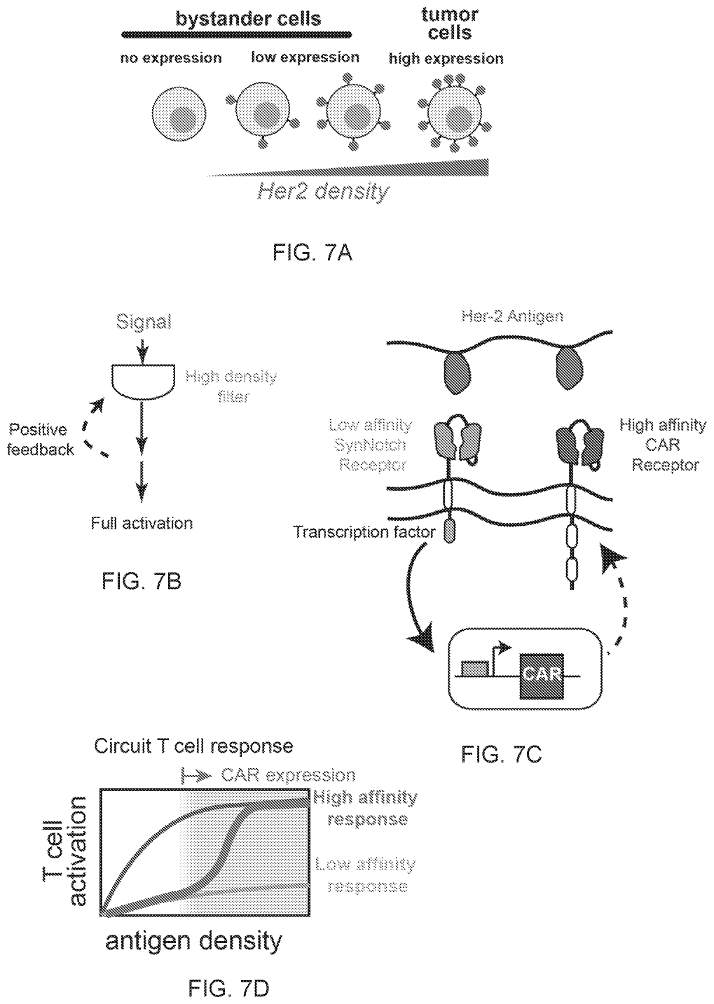

Several synNotch/CAR circuits have been designed and expressed. The ability of such circuits to achieve antigen density sensing has also been tested in vitro and in vivo. The circuits of this example use a synNotch receptor to control the expression of a CAR recognizing the Her2 ligand. FIG. 7A-7D demonstrates the design for engineered T cells that recognize and discriminate cancer cells from normal/bystander cells based on target antigen density. FIG. 7A depicts this concept as it relates to cancer cells expressing the antigen HER2, where the majority of CAR T cell antigen targets (in this example Her2) are also present at low levels in normal tissue. To make treatment with CAR T cells more broadly applicable, it is desirable that engineered T cells discriminate normal tissue from diseased based on antigen density. FIG. 7B schematically depicts a circuit that integrates a high antigen density filter and positive feedback to control T cell activation. FIG. 7C provides a cartoon of an affinity tuned two-step circuit. A low affinity SynNotch receptor against a Her-2 antigen driving the expression of a high affinity CAR against the same antigen induces a strong T cell activation only when high Her2 density is encountered. FIG. 7D demonstrates the outcome of the circuit illustrated in FIG. 7C. At low Her2 densities, the response, if any, is low; however, at the threshold density the CAR expression triggers an amplified strong output.

The constructs are introduced into T cells via standard lentiviral infection, and the T cells are sorted for receptor expression using flow cytometry (anti-myc stain). Candidate receptors have been screened for expression and activation response in the human Jurkat T cell line and human primary CD4+ and CD8+ T cells from several donors, using standard flow-based assays (including e.g., CD69 Activation, T cell proliferation) and cytokine assays (including e.g., IL-2, INF-γ).

The best performing candidate circuits were further evaluated in in vivo mouse models. The growth curves of tumors containing engineered target cells have been evaluated to measure baseline tumor growth in the model system. Then either conventional CAR T cells or novel antigen-density sensing circuit engineered T cells were administered to NSG immunocompromised mice containing bilateral tumors, in which one side contains a low density Her2 K562 tumor and the other side contains a higher density Her2 K562 tumor. The engineered T cells are delivered by injection into the tail vein, and allowed to traffic freely to either tumor. Tumor size was monitored using calipers over the course of 4 weeks, and the differential clearance of the high and low density tumors was tracked.

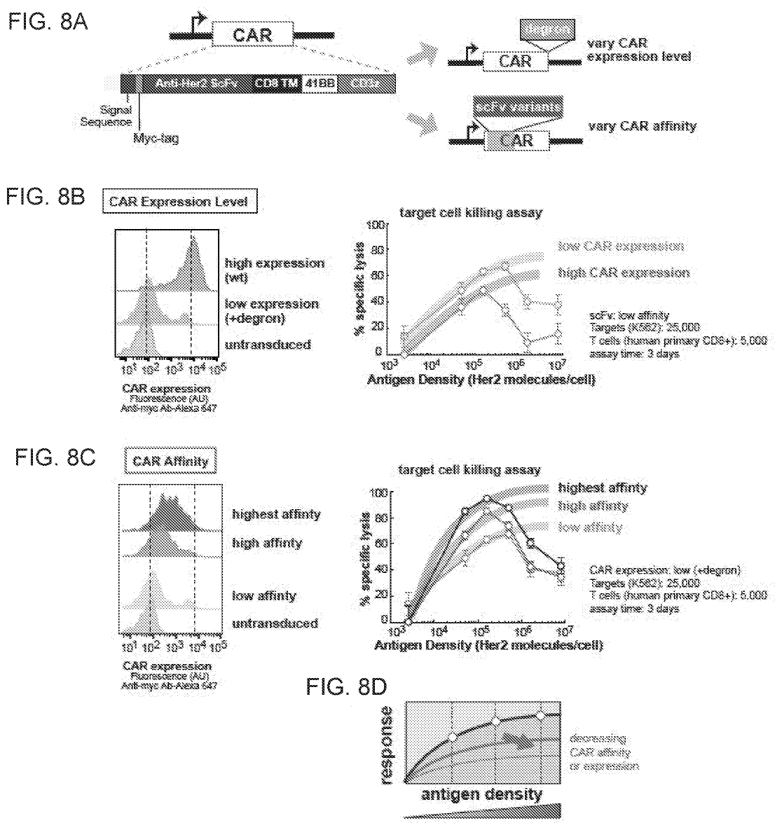

Selective antigen density target cell killing In vitro by affinity tuned SynNotch-CAR T cells was further investigated using an anti-Her2 CAR (see design provided in FIG. 8A). Anti-Her2 4D5 antibodies that were mutated at the recognition domain to result in a series of variants with reduced Her2 affinity were used to build affinity tuned SynNotch-CAR circuits (see Carter et al., PNAS (1992) 89:4285-4289 and Liu et al., Cancer Res (2015) 75(17):3596-3607). A target cell line series with varying Her2 density was constructed. Altering CAR expression level or affinity yields modest linear changes in antigen density sensitivity. T cells expressing varying levels of receptor were obtained by fusing a degron tag to the CAR. CAR affinity was altered by changing the scFv domain. FIG. 8B shows the effect of changing CAR expression levels on antigen density dependent cell killing. FACs plots on left show CAR expression distribution in human primary CD8+ T cells, with and without degron tag. Plot on right shows antigen density dependence of target cell killing. Effector:target cell ratios were kept low (1:5) so that there was always an excess of target cells. Biphasic response is likely due to trogocytosis (T cell uptake of the Her2 antigen, leading to T cell fratricide); transparent lines are drawn based on inspection. FIG. 8C shows the effect of changing CAR affinity on antigen density dependent cell killing. FACS plots on left show CAR expression distribution of each affinity CAR in human primary CD8+ T cells. Plot on right shows antigen density dependence of target cell killing. Effector:target cell ratios were kept low (1:5) so that there was always an excess of target cells. Biphasic response is likely due to trogolcytosis (T cell uptake of the Her2 antigen, leading to T cell fratricide); transparent lines are drawn based on inspection. The percentage of specific lysis was determined using flow cytometry by counting the number of target cells after 3 days relative to a co-culture in the presence of untransduced T cells. Overall, changing CAR affinity or expression leads to linear changes in antigen density response curves (FIG. 8D).

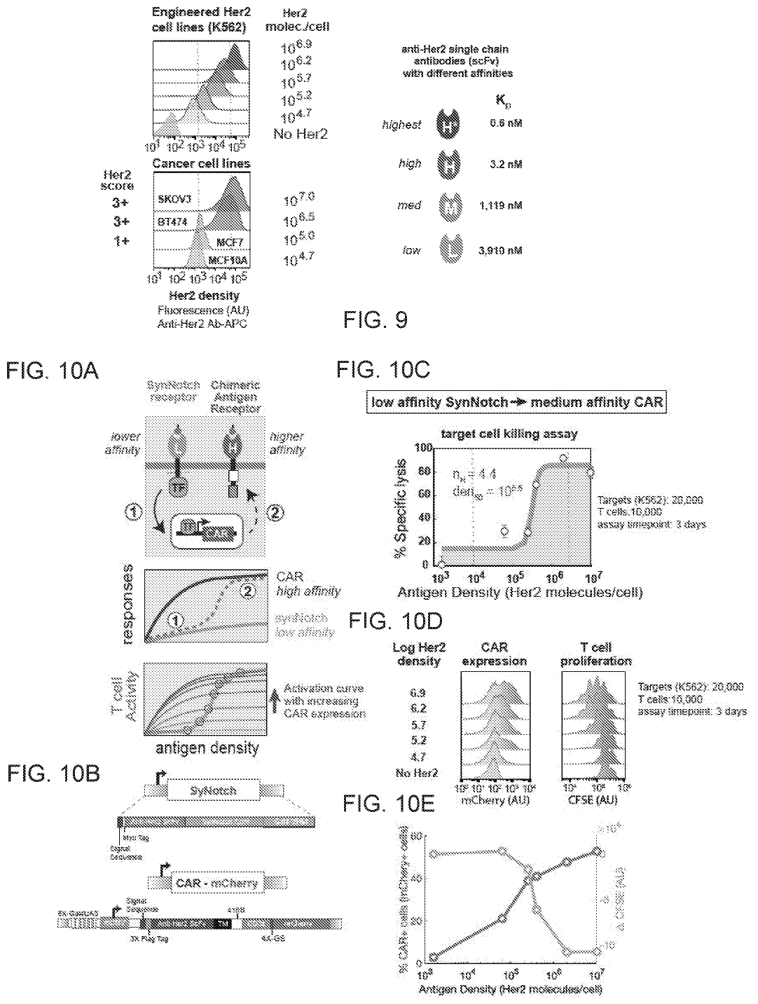

As shown in FIG. 9 a series of Her2 expressing cell lines, stable K562 tumor cells that express different levels of Her2, were constructed to allow for systematically measuring density discrimination. These engineered series of target cells span a range of densities that correlate with those observed in Her2 overexpressing tumor cells (e.g. SKBR3, SKOV3). To quantitatively assay antigen density sensing, stable cell-lines of K562 cells expressing the shown densities of the cancer associated antigen Her2 were engineered. These cell lines express Her2 levels that span those of normal and tumor cell lines (bottom plot; Her2 pathology score is shown on left of plot). To construct different Her2 sensing systems, a series of anti-Her2 single chain antibodies with affinities spanning a range of over 1,000-fold were utilized.

A two-step low-to-high affinity recognition circuit yields ultrasensitive antigen density sensing. A schematic of the components expressed in the circuit is provided in FIG. 10A. A synNotch receptor detects antigen (Her2) with low affinity. This synNotch receptor, when activated, induces expression of a high affinity CAR. In principle, cells with this circuit combine two different response curves—early on the cell will be dominated by the low affinity synNotch activation, and later by the high affinity CAR activity. T cell activity is predicted to show a robust sigmoidal response curve, because as antigen density increases, this leads to a gradual increase in CAR expression, transiting between the series of linear response curves shown in purple. For each antigen density, the black circles show intersection with the linear response curve for the steady-state level of CAR expression induced at that antigen density. In FIG. 10B, to track CAR expression, a mCherry protein was fused to the C-terminus of the anti-Her2 CAR construct. FIG. 10C shows in vitro cell killing curve as a function of target cell antigen density. Human primary CD8+ T cells expressing a two-step circuit, in which the low affinity synNotch receptor induces expression of the medium affinity CAR, were used. Solid line is fit to a hill equation. Dotted black lines show Her2 densities corresponding to low (+1) or high tumor (+3) cell lines. The percentage of specific lysis was determined using flow cytometry by counting the number of target cells after 3 days relative to a co-culture in the presence of untransduced T cells. FIGS. 10D-10E show FACS distributions and quantitation for CAR expression and T cell proliferation measured as a function of target cell Her2 density (at 3 days) for the circuit T cells. As shown, significant CAR expression and T cell proliferation is only observed at densities of >10′.

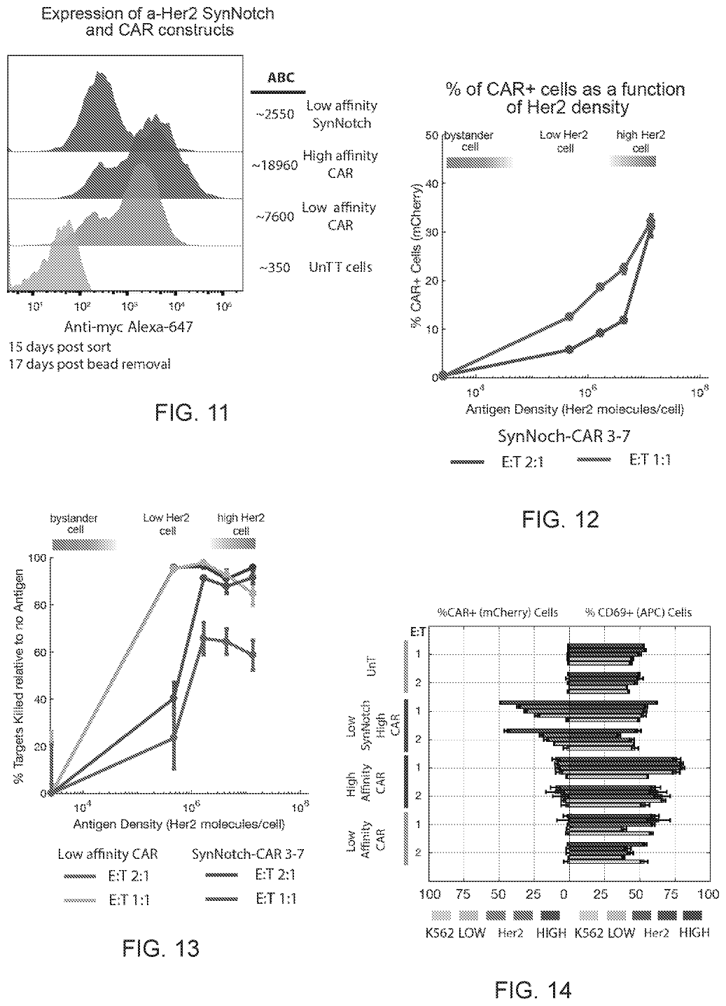

FIG. 11 demonstrates the expression of Her2 specific SynNotch-CAR circuits in primary CD8+ T cells. The plot shows the expression of low and high affinity CARs and Low affinity SynNotch receptors in CD8+ cells. Fluorescence intensity and the Antibody binding capacity (ABC, number of receptors/cell) was been calibrated as indicated. FIG. 12 shows that low affinity SynNotch receptors gate the CAR expression in an antigen density dependent manner. Co-culture assays of T cells and targets at 2:1, 1:1 ratio show that T cells bearing low affinity receptors are able to tune CAR expression as a function of the target antigen density. FIG. 13 show that, unlike conventional CARs alone, the affinity tuned SynNotch-CAR circuits kill target cells (see flow cytometry counts) discriminating between cells with different antigen levels. For comparison the low affinity (blue lines) and SynNotch-CAR (low-high) at a 2:1 and 1:1 effector to target ratio data are also shown. The co-culture assays were carried for 3 days starting with 25K target cells. FIG. 14 provides a summary of CAR expression and T cell activation in the various conditions tested.

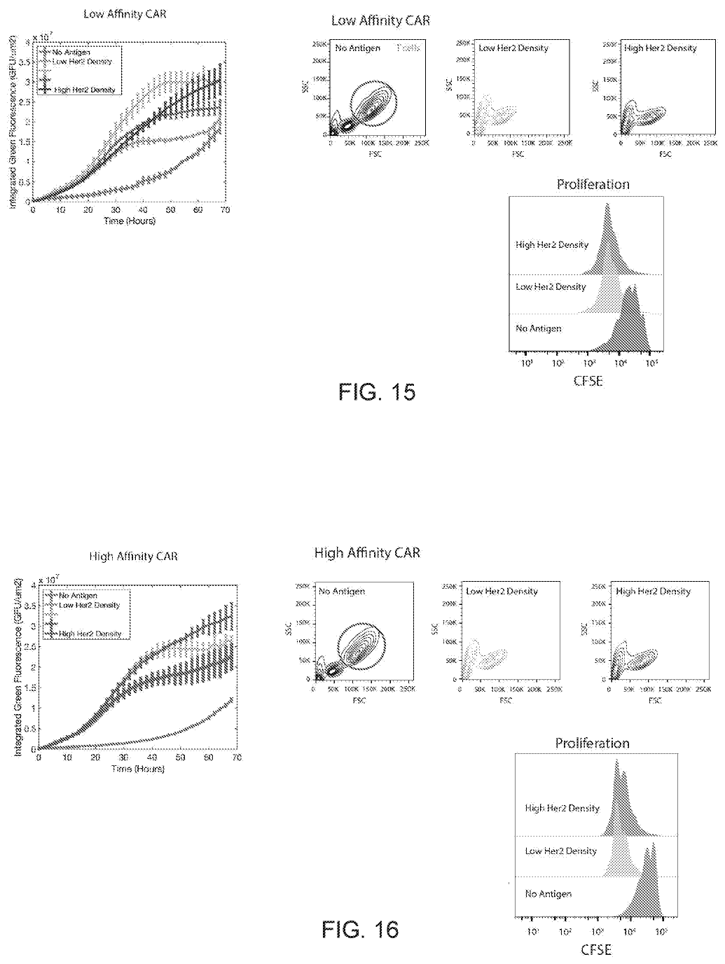

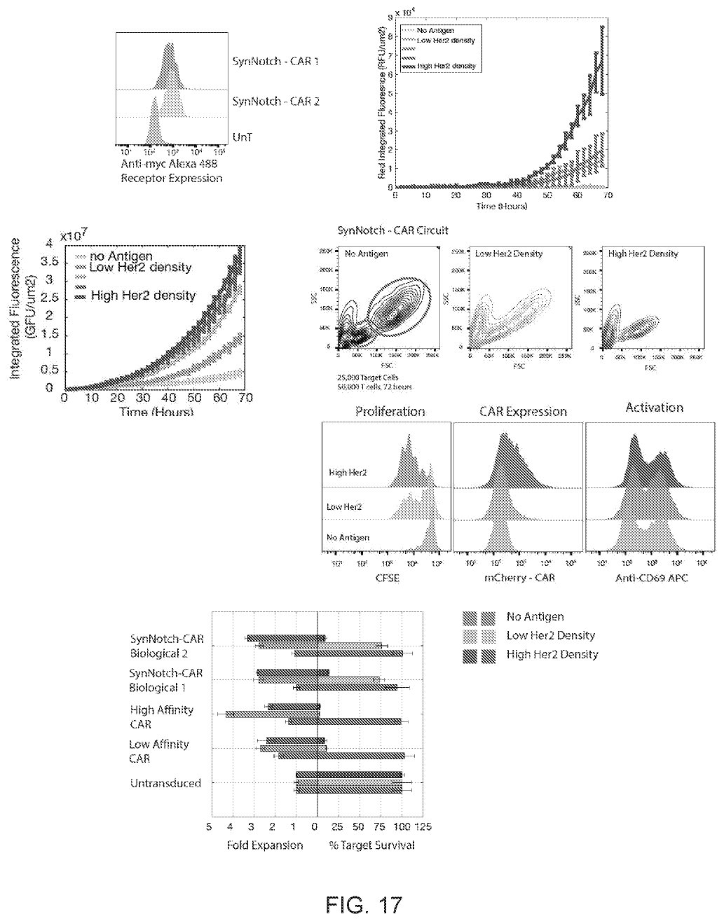

FIGS. 15 and 16 show that low (FIG. 15) and high (FIG. 16) affinity Her2 CARs do not discriminate between low and high density targets. For FIGS. 15 and 16 microscopy-based assay, 500 target cells were plated for 48 hours before adding 1000 T cells. The killing was determined up to 68 hours and caspase green dye was used for staining. For FIGS. 15 and 16 flow cytometry killing assay, 15000 target cells and 15000 T-cells were used and killing was determined at 72 hours. These studies have confirmed that when using a fixed affinity standard CAR T cell, the administered cells kill both high and low level Her2 expressing tumors with equal efficiency. However, as shown in FIG. 17, synNotch-CAR circuits are capable of discriminating between low and high antigen density targets. Parallel mouse experiments with the cells containing the antigen-density sensing two receptor circuit show improved discrimination of target cells based on antigen density. For FIG. 17 microscopy-based assay, 500 target cells were plated for 48 hours before adding 1000 T cells. The killing was determined up to 68 hours and caspase green dye was used for staining.

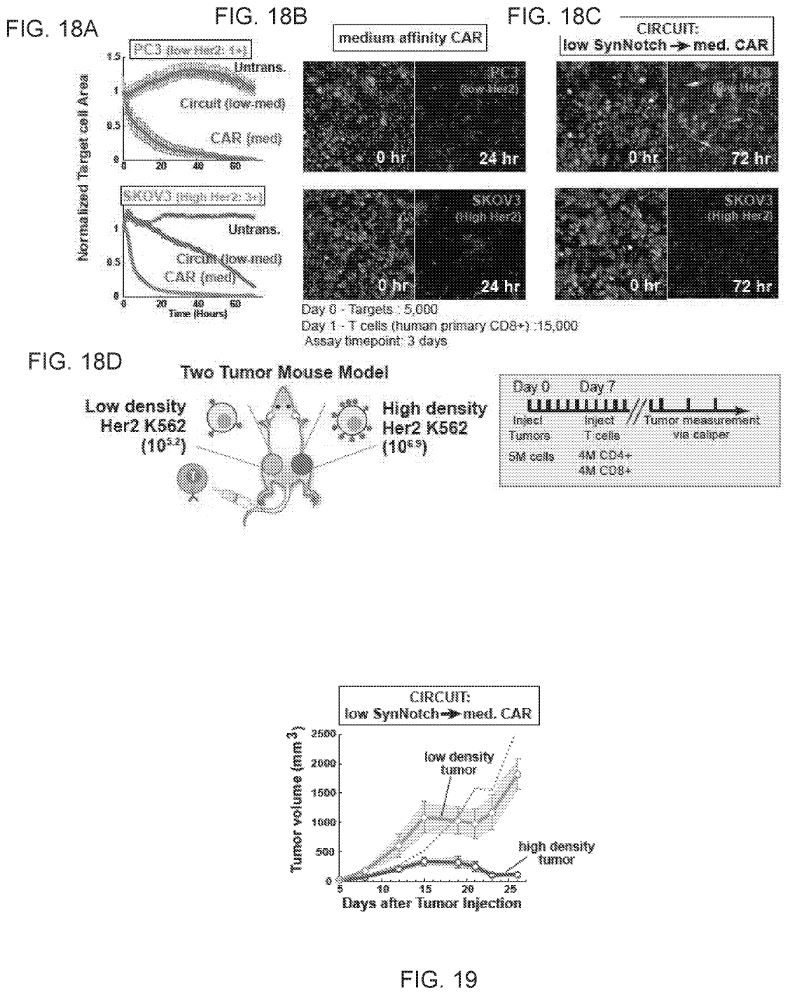

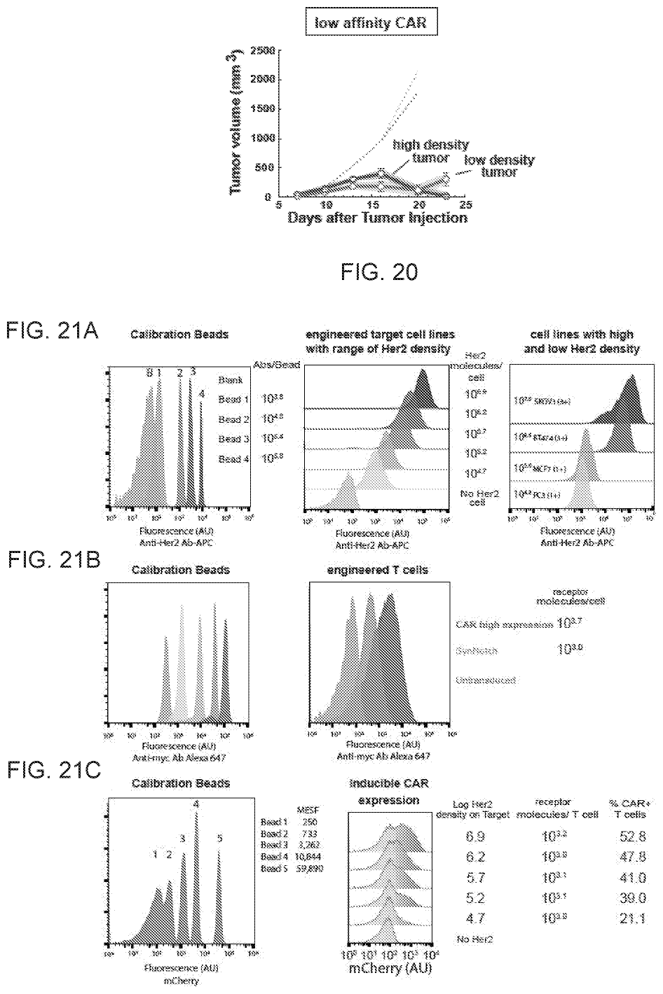

For example, FIG. 18-20 demonstrate selective antigen density target cell killing in vivo by affinity tuned SynNotch-CAR T cells. FIG. 18 schematically depicts a two-tumor mouse model used to test antigen density sensing in vivo. 5 million low and high density K562 cells were injected subcutaneously in the left and right flanks of NSG mice. Primary human CD4+ and CD8+ engineered T cells were injected intravenously 7 days after tumor injection. FIG. 19 provides graphs showing low and high Her2 tumor volumes for mice treated with High affinity CAR T cells and untransduced control T cells. High affinity CAR T cells failed to discriminate between low and high density Her2 tumors (n=5 mice, error bars are SEM). FIG. 20 provides graphs showing low and high Her2 tumor volumes for mice treated with affinity tuned Low-High SynNotch-CAR T cells and untransduced control T cells. SynNotch-CAR T cells target the high density tumors exclusively, the low density Her2 tumors grew at the same rate as in mice treated with untransduced control T cells (n=5 mice, error bars are SEM).

FIGS. 21A-21C provide determination of antigen density and receptor expression from fluorescence intensity. Antigen density and receptor expression were determined by quantitative flow cytometry. FIG. 21A (left) provides representative flow cytometry histograms showing the fluorescence intensity of Quantum Symply Cellular anti-Mouse IgG beads (Bang Laboratories 815) stained with anti-Her2 APC antibody. The manufactured antibody binding capacity of each bead population is indicated to the right in top panel. FIG. 21A (center) shows representative flow cytometry histograms of engineered K562 Her2-BFP cell lines stained with anti-Her2 APC antibody. The geometric mean of each population and the calibration curve built from data shown in the left panel was used to determine the number of Her2 molecules per cell in each population. FIG. 21A (right) shows representative flow cytometry histograms showing the fluorescence intensity of cancer cell lines expressing a range of Her2 densities. The density of Her2 molecules/cell and their classification as scored by ASCO-CAP scoring guidelines is shown to the left. FIG. 21B (left) shows, similar to FIG. 21A, for beads stained with anti-myc Alexa 647. FIG. 21B (center) shows engineered T cells expressing either a constitutive CAR or SynNotch receptor were stained with anti-myc Alexa 647. The number of receptors per T cell populations was determined as described above. FIG. 21C (left) shows representative flow cytometry histograms of beads showing fluorescence intensity equivalent to the indicated number of soluble mCherry molecules (MESF). FIG. 21C (center) shows representative histograms showing the fluorescence intensity of T cells co-cultured with K562 Her2-BFP target cells for 3 days. The geometric mean of the positive population and the corresponding calibration curve was used to determine the number of inducible CAR molecules per cell in each population. The percentage of CAR positive cells was determined using the population comparison platform in FlowJo V10 and it is reported as % SE Dymax. Briefly, it normalizes the data to a unit scale to protect against outliers, and factors in the distribution of the data.

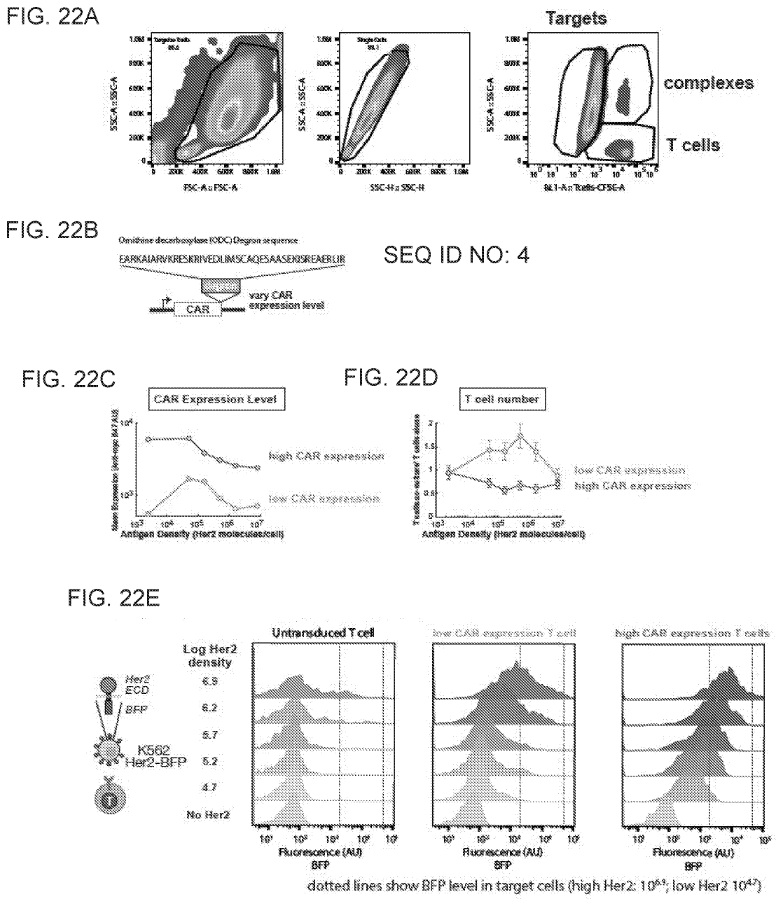

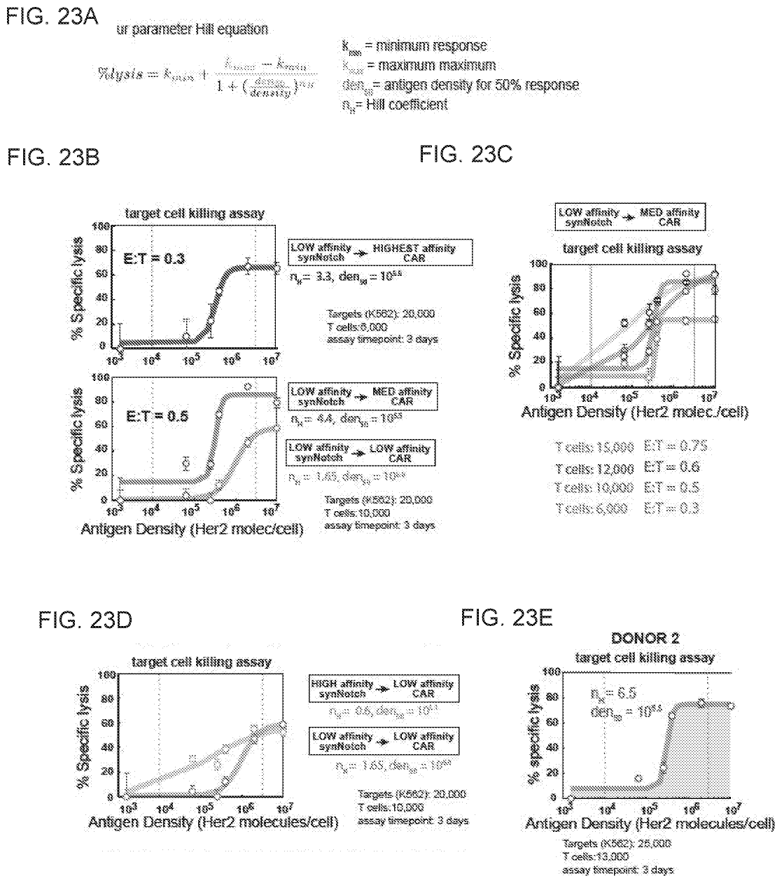

FIGS. 22A-22E provide killing assay gating scheme, CAR T cell receptor expression and trogocytosis analysis. FIG. 22A provides details on gating scheme utilized to analyze killing assays by flow cytometry. Samples were first gated using a live-dead cell stain dye, then using forward and side scattering profiles to select single cells and finally using the CFSE celltrace fluorescence to separate T cells from K562-Her2 targets. T cell-Target complexes were excluded from the analysis. FIG. 22B shows construct design to obtain low expression levels of anti-Her2 CARs. A degron sequence corresponding to the C-terminal region of mouse Ornithine decarboxylase (termed cODC) (EARKAIARVKRESKRIVEDLIMSCAQESAASEKISREAERLIR) (SEQ ID NO:4) was fused to the original CAR constructs. FIG. 23C shows T cell CAR expression levels as a function of target antigen density after 3 days of co-culture. T cells expressing high levels of CARs show lower CAR levels as the target antigen density increases. T cells expressing low CAR levels upregulate their CAR expression when co-cultured with low density target cells but show lower CAR expression levels as the target antigen density increases. FIG. 22D provides ratio of T cell counts when cultured either alone or with K562-Her2 targets after 3-days of co-culture. The T cells numbers in the co-culture for T cells expressing high levels of CAR diminish as the target antigen density increases. In contrast, T cells expressing low CAR levels show higher numbers when co-cultured with low Her2 targets. A potential explanation for the T cell counts presented in D and the biphasic behavior of the target killing results is that trogocytosis increases as a function of target antigen density. FIG. 22E shows representative FACS histograms of BFP fluorescence intensity shown by T cells after 3 days of culture with K562-Her2 (BFP-tagged) targets. The BFP fluoresce intensity on T cells increases as the Her2 density on the targets increases. T cells expressing high levels of anti-Her2 CAR show higher BFP levels than T cells expressing low levels of anti-Her2 CAR. Dotted lines show the BFP fluorescence intensity in the high and low Her2-BFP target cells.

FIGS. 23A-23E show effects of receptor affinity and T cells dosage on two-step circuit function. FIG. 23A provides four parameter Hill equation utilized to fit the killing response curves as a function of antigen density of two-step circuits tested in this study. The parameters are colored coded and indicated to the right of the equation. The hill coefficient (nH) and antigen density for the half maximal activity values are indicated for each killing curve. FIG. 23B shows target cell killing response curves for T cells expressing other two-step circuits. A low affinity SynNotch receptor was used in these circuits. Different affinity CAR receptors were tested. All circuits show ultrasensitive Her2 sensing. The low affinity SynNotch to low affinity CAR circuit showed a higher antigen threshold than the other two designs. Accordingly, all low affinity synNotch circuits show ultrasensitive Her2 sensing. FIG. 23C provides Target cell killing response curves for T cells expressing low affinity SynNotch to medium affinity CAR circuit at different effector to target (E:T) ratios. Ultrasensitivity is best observed at low E:T ratios. Therefore, ultrasensitivity is best measured at low E:T ratio (limiting T cells, excess targets). If T cells are in excess, ultrasensitivity is reduced. FIG. 23D provides target cell killing response curves for T cells expressing two-step circuits where the SynNotch affinity was changed. High affinity SynNotch to low affinity CAR T cells showed reduced ultrasensitivity and lower Her2 density threshold than the low affinity SynNotch to low affinity CAR. Thus, in the two-step synNotch-CAR circuit, increasing affinity of synNotch reduces ultrasensitivity. FIG. 23E shows target cell killing response curve for T cells expressing low affinity SynNotch to medium affinity CAR circuit from a different donor.

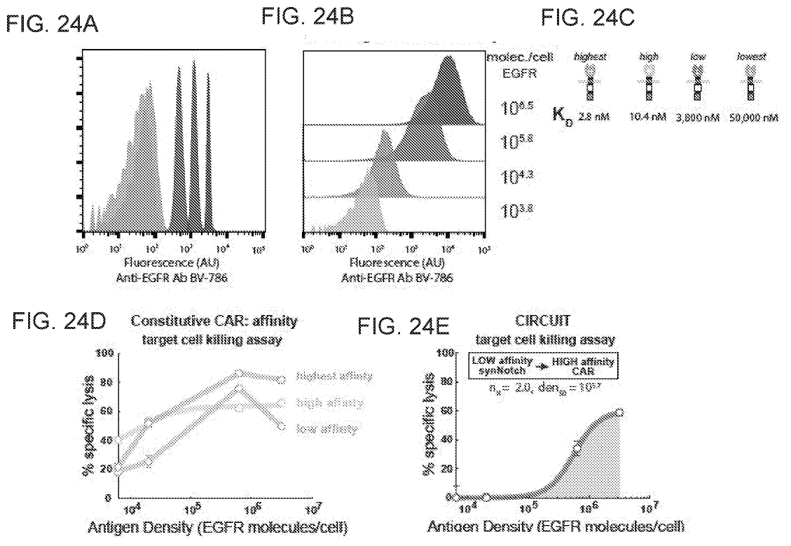

FIGS. 24A-24E show T cells expressing a two-step circuit low-to-high SynNotch-CAR affinity recognition circuit yield ultrasensitive antigen density sensing against EGFR engineered cells. FIG. 24A provides representative flow cytometry histograms showing the fluorescence intensity of Quantum Symply Cellular anti-Mouse IgG beads (Bang Laboratories 815) stained with anti-EGFR BV786 antibody. FIG. 24B shows representative flow cytometry histograms of engineered K562 EGFR cell lines stained with anti-EGFR BV786 antibody. The geometric mean of each population and the calibration curve built from data shown in the A was used to determine the number of EGFR molecules per cell in each population. FIG. 24C shows series of ScFv and nanobodies utilized to build two-step SynNotch to CAR circuits. Their reported affinities are indicated. FIG. 24D provides target cell killing activity as a function of EGFR antigen density for T cells expressing CARs of indicated affinities. The E:T and assay time point is indicated below the plot. Targets (K562): 25000; T cells (human primary CD8+): 10000; assay time point: 3 days. FIG. 24E provides target cell killing activity as a function of EGFR antigen density for T cells expressing a low affinity SynNotch to high affinity CAR circuit. Targets (K562): 25000; T cells (human primary CD8+): 3000; assay time point: 3 days.

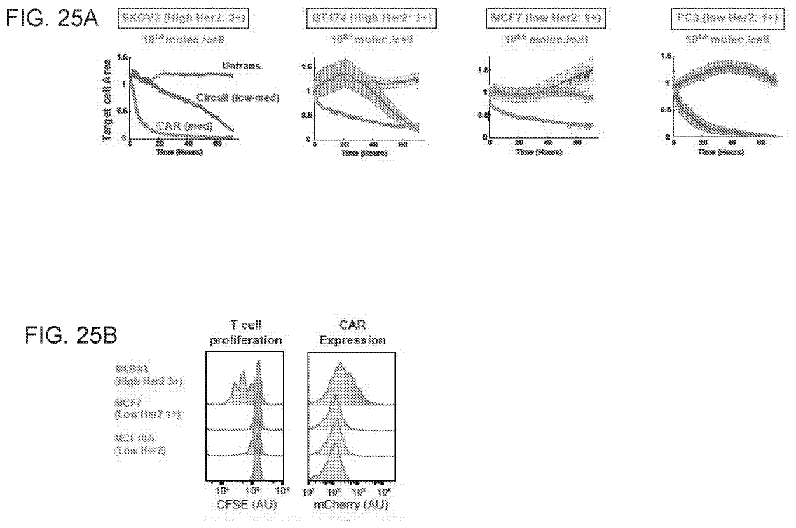

FIGS. 25A-25B show that low affinity SynNotch to medium affinity CAR T cells show antigen density activity against several Her2 positive cancer cell lines. FIG. 25A provides in vitro target cell area over time. Cancer lines with several Her2 densities were co-cultured with human primary CD8+ T cells expressing either a two-step circuit (low affinity to medium affinity CAR) or a medium affinity CAR. Gray lines correspond to the target cell area in the presence of Untransduced T cells. Solid lines show the average target area and error bars show the standard deviation (n=3). The mean Her2 density and classification is indicated for each cancer line. Targets Day 0: 5000; T cells (human primary CD8+) Day 1: 15000; assay time point: 3 days. Low density Her2 cells (PC3) were labelled with green cell trace (CFSE). Some photolabelling was observed after 40 hours when the field of view saturates with cells. FIG. 25B shows representative FACS plots of inducible CAR expression and T cell proliferation for T cells co-cultured with cancer cell lines expressing high and low Her2 densities. The E:T and assay time point are indicated below the histograms. Targets (cancer cells) Day 0: 10000; T cells (human primary CD8+) Day 1: 25000; assay time point: 3 days.

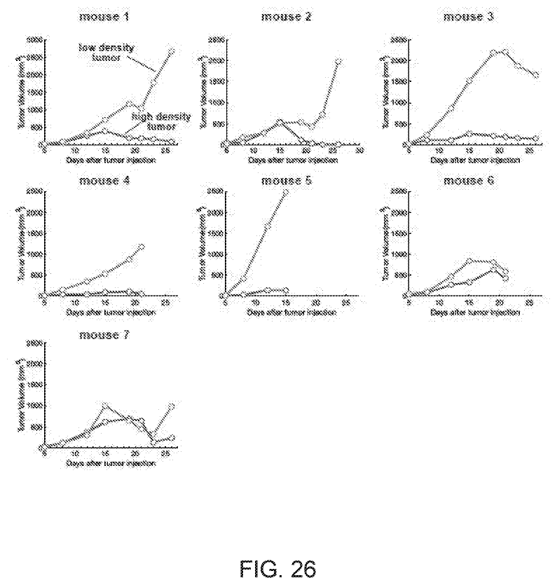

FIG. 26 shows tumor volume measurements for individual mice treated with T cells expressing low affinity SynNotch to medium affinity CAR circuit. Tumor volume data for individual mice treated with T cells expressing a low affinity SynNotch to medium affinity CAR circuit. The dark purple lines correspond to the high Her2 K562 tumor whereas the light pink lines correspond to the low Her2 K562 tumor.

These studies confirm that cells containing the antigen-density sensing two receptor circuit show improved discrimination of target cells based on antigen density in vivo.

### Materials and Methods

CAR Receptor Design: Chimeric Antigen Receptors were built by fusing Anti-Her2 antibodies with different affinities (see Liu, et al., Cancer Research 75(17), 2015; the disclosure of which is incorporated herein by reference in its entirety), to a CD8 transmembrane, 41BB co-stimulation domain, CD3ζ intracellular signaling domain. All CAR receptors contain an n-terminal CD8a signal peptide (MALPVTALLLPLALLLHAARP; SEQ ID NO:1) for membrane targeting and a myc-tag (EQKLISEEDL; SEQ ID NO:2) for easy determination of surface expression with α-myc Alexa 647 or 488 antibodies (Cell-Signaling, catalog #2233). The receptors were cloned into a modified pHR′SIN:CSW vector containing a SFFv promoter for all primary T cell experiments. The constructs were cloned via In fusion cloning (Clontech, catalog #ST0345). To obtain low expression levels of CARs, a degron sequence corresponding to the C-terminal region of mouse Ornithine decarboxylase (termed cODC) (EARKAIARVKRESKRIVEDLIMSCAQESAASEKISREAERLIR) (SEQ ID NO:4) was fused to the CD3z signaling domain following a (G₄S)₃ (SEQ ID NO:9) linker. The anti-Her2 antibody variants' scFv sequences are provided below:

anti-Her2 antibody variant_ HIGH scFv sequence:
(SEQ ID NO: 5)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSSGS;
anti-Her2 antibody variant_ INTERMEDIATE scFv
sequence:
(SEQ ID NO: 6)
DIQMTQSPSSLSASVGDRVTITCRASQDVNTAVAWYQQKPGKAPKLLIYS
ASFLESGVPSRFSGSRSGTDFTLTISSLQPEDFATYYCQQHYTTPPTFGQ
GTKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSSGS
anti-Her2 antibody variant_ LOW scFv sequence:
(SEQ ID NO: 7)
DIQMTQSPSSLSASVGDRVTITCRASQDVNTAVAWYQQKPGKAPKLLIYS
ASFLESGVPSRFSGSRSGTDFTLTISSLQPEDFATYYCQQHYTTPPTFGQ
GVKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSSGS;
and
anti-Her2 antibody variant_ LOWEST scFv sequence:
(SEQ ID NO: 8)
DIQMTQSPSSLSASVGDRVTITCRASQDVNTAVAWYQQKPGKAPKLLIYS
ASFLESGVPSRFSGSGSGTDFTLTISSLQPEDFATYYCQQHYTTPPTFGQ
GVKVEIKRTGSTSGSGKPGSGEGSEVQLVESGGGLVQPGGSLRLSCAASG
FNIKDTYIHWVRQAPGKGLEWVARIYPTNGYTRYADSVKGRFTISADTSK
NTAYLQMNSLRAEDTAVYYCSRWGGDGFYAMDVWGQGTLVTVSSGS.

SynNotch Receptor and Response Element Construct Design: SynNotch receptors were built by fusing Anti-Her2 antibodies (Liu, et al., supra), to the mouse Notch1 minimal regulatory region (I1e1427 to Arg1752) and Gal4 DNA binding domain (DBD) VP64. All synNotch receptors contain an n-terminal CD8a signal peptide (MALPVTALLLPLALLLHAARP; SEQ ID NO:1) for membrane targeting and a myc-tag (EQKLISEEDL; SEQ ID NO:2) for easy determination of surface expression with α-myc Alexa 647 or 488 (Cell-Signaling, catalog #2233). The receptors were cloned into a modified pHR′SIN:CSW vector containing a PGK promoter for constitute expression of the SynNotch receptor. The same vector was also modified to have the response element within the same vector. Five copies of the Gal4 DBD target sequence (GGAGCACTGTCCTCCGAACG; SEQ ID NO:3) were cloned 5′ to a minimal CMV promoter. For all inducible CAR vectors, the CARs were tagged c-terminally with and mCherry domain for tracking of CAR expression either by microscopy or flow cytometry. All constructs were cloned via In fusion cloning (Clontech, catalog #ST0345).

Generation of Her2-K562 Cells with different Her2 expression levels: The cancer cell lines used were K562 myelogenous leukemia cells (ATCC #CCL-243). K562s were lentivirally transduced to stably express human Her2 extracellular and transmembrane domains (residues 23-675) fused to a BFP. The construct contains an N-terminal CD8a signal peptide (MALPVTALLLPLALLLHAARP; SEQ ID NO:1) for membrane targeting. Her2 levels were determined by staining the cells with α-Her2 APC (Biolegend, catalog #324408) or PE (BD Biosciences, catalog #340552) and sorted using a Cell Sorter FACS Aria II. The absolute amount of Her2 molecules on the cell surface was estimated by comparing the fluorescence intensity in the cell population with that of beads coated with a fixed number of molecules (Quantum Simple Cellular—Anti Mouse IgG—Bang Laboratories, Inc and Anti-Her2 Clone NEU 24.7—PE, BD Biosciences). The Her2-BFP construct was expressed under the control of the SFFV promoter. Overexpression of Her2 is consistent with the amplified levels found in +3, +2 and +1 tumors as scored by ASCO-CAP scoring guidelines (FIG. 1B). All engineered K562 cell lines were subcultured in IMDM media supplemented with 10% FBS and gentimicin.

Assessment of Affinity tuned SynNotch-CAR T cell Cytotoxicity: CD8+ synNotch-CAR affinity tuned T cells were stimulated for 72 hours with target cells expressing the indicated antigens. The level of specific lysis of target cancer cells was determined by comparing the fraction of target cells alive in the culture compared to treatment with untransduced T cell controls. Cell death was monitored by a caspase 3/7 dye by microscopy (Incucyte, Essen Biosciences) or by flow cytometry by shifting of the target cells out of the side scatter and forward scatter region normally populated by the target cells.

Proliferation and CD69 Staining: Primary CD8+ SynNotch-CAR T cells were stained with a Celltrace CFSE dye following manufacturer instructions (Thermo Fisher, catalog C34554) and stimulated with the different Her2 cancer cell lines, as described above, for 72 hours. The T cells were also collected and stained with α-CD69 APC (Biolegend, catalog #310910) to determine if they were activated and analyzed by flow cytometry.

Engineered EGFR Cell Lines: A series of EGFR amplified lines was obtained using a construct that expresses the extracellular region (AA 1-645) of EGFR and transmembrane region of PDGFR (AA 512-561). All cell lines were stained and sorted for expression of transgenes using an anti-EGFR BV786 (BD Bioscience 742606) antibody. Engineered K562 cell lines were subcultured in IMDM media supplemented with 10% FBS (Fetal bovine serum) and gentimicin.

Cancer Cell lines: All cancer cell lines used in this study were purchased from the indicated vendors. Cells were cultured to confluence in the indicated media supplemented with 10% FBS. At each passage, cells were washed with PBS (Phosphate-buffered saline at 37° C.) and TrypLE (ThemoFisher Scientific 12604021) was added. Flasks containing the cells were allowed to sit at 37° C. until the cells detached, typically 5 to 10 min. Fresh culture medium was added to quench the TrypLE and cells were resuspended and plated in new flasks and in fresh culture medium. PC3 cells (ATCC CRL-1435) were cultured in F-12K medium, SKOV3 cells (ATCC CRL-HTB77) in McCoy's 5a medium, MCF7 cells (ATCC CRL-HTB22) in DMEM medium, BT474 cells (ATCC CRL-HTB20) in RPMI medium and MCF10-A (ATCC CRL-10317) in DMEM/F-12 medium supplemented with 5% horse serum, cholera toxin to a final concentration of 1 ng/mL; human insulin to a final concentration of 10 ug/mL; epidermal growth factor to a final concentration of 10 ng/mL; and hydrocortisone to a final concentration of 0.5 ug/mL.

Determination of protein copy number per cell: Antigen density per target cell was determined by quantitative flow cytometry. 1×10⁵ cells of each population were stained with either Anti-Her2 APC (Biolegend 324407), Anti-EGFR BV786 (BD Biosciences 742606) or Anti-myc Alexa 647(CellSignaling 2233S) antibody for 30 min on ice (n=3). Cells were washed twice with PBS and resuspended in PBS for analysis in an Attune NxT Flow Cytometer. The geometric mean of each target population was determined after gating the cells by their size (side scatter and forward scatter region) and selecting the full width at half maximum (FWHM) of the population in the corresponding fluorescent channel. A standard curve was built using Quantum Symply Cellular anti-Mouse IgG beads (Bang Laboratories 815) stained with the same antibody than the target cells. For each cell line, the number of molecules per cell was determined using the standard curve and the geometric mean of each target population. Similarly, to determine the expression amounts of inducible CAR, mCherry flow cytometer calibration beads (Takara Bio 632595) were used.

Primary Human T Cell Isolation and Culture: Primary CD4+ and CD8+ T cells were isolated from blood of anonymous donors by negative selection (STEMCELL Technologies #15062 and #15063). T cells were cryopreserved in RPMI-1640 (UCSF cell culture core) with 20% human AB serum (Valley Biomedical, #HP1022) and 10% DMSO (dimethyl sulfoxide). T cells were cultured in human T cell medium consisting of X-VIVO 15 (Lonza #04-418Q), 5% Human AB serum, and 10 mM neutralized N-acetyl L-Cysteine (Sigma-Aldrich #A9165) supplemented with 30 units/mL IL-2 (NCI BRB Preclinical Repository) for all experiments.

Lentiviral Transduction of Human T Cells: Pantropic VSV-G pseudotyped lentivirus was produced by transfecting Lenti-X 293T cells (Clontech #11131D) with a pHR′SIN:CSW transgene expression vector and the viral packaging plasmids pCMVdR8.91 and pMD2.G using Fugene HD (Promega #E2312). Primary T cells were thawed and after 24 hr in culture, were stimulated with Human T-Activator CD3/CD28 Dynabeads (Life Technologies #11131D) at a 1:3 cell:bead ratio. After 48 hr, viral supernatant was harvested and added to primary T cells. T cells were exposed to the virus for 24 hr. At day 5 after T cell stimulation, the Dynabeads were removed. T cells were stained and sorted to obtain homogenous expression levels. For the SynNotch to CAR circuits, T cells expressing CAR were removed by cell sorting. T cells were expanded for at least 9 days when they were rested and could be used for killing assays.

In vitro T Cell Cytotoxicity Assessment: CD8+ Primary human T cells expressing either anti-Her2 CAR or anti-Her2 SynNotch-CAR circuits were co-cultured for 72 hr with targets expressing different Her2 densities in complete human T cell medium and placed at 37° C., 5% CO₂ incubator. For all in vitro T cell killing assays against engineered K562-Her2 cells T cells were stained with celltrace CFSE dye (Thermo Fisher Scientific C34554) and co-cultured in round bottom 96-well tissue culture plates at the indicated effector to target ratios. The cells were centrifuged for 1 min at 400×g to favor effector to target interactions, and the cultures were analyzed at 72 hr for specific lysis of target tumor cells, T cell proliferation and CAR expression by flow cytometry. T cells were identified by the celltrace dye and target cells were identified by size. The level of specific lysis of target cancer cells was determined by comparing the number of target cells alive in the culture compared to treatment with untransduced T cell controls. Cell death was monitored by a live-dead cell stain and by shifting of the target cells out of the side scatter and forward scatter region normally populated by the target cells. All flow cytometry was performed using BD LSR II or Attune NxT Flow Cytometers and the analysis was performed in FlowJo software (TreeStar) and Matlab.

Imaging of T cell Cytotoxicity-2D cultures: For all in vitro T cell killing of Her2 expressing cancer lines, 5×10³ target cells were stained with a celltrace dye, cultured overnight in a flat bottom 384-well tissue culture plate in their indicated medium and placed at 37° C., 5% CO₂ incubator. After 1 day, 1.5×10⁴ T cells were stained and added to the flat bottom 384-well tissue culture plate and the co-cultures were imaged every hour for 3 days. Two fields per well were imaged using the 20× objective on a PerkinElmer Opera Phenix High Content Screening System and the images were analyzed using the associated Harmony Office Software. Data was summarized as the sum of the normalized area occupied by target cells and presented as mean±SEM.

Statistical Analysis and Curve Fitting: Data is presented as means±standard error of the mean (SEM) or means±standard deviations (SD) as indicated in the figure legends. The target cell killing data for the SynNotch to CAR circuits was fitted to a four parameter Hill equation using the curve fitting toolbox in MatLab.

In vivo Mouse Models: All mouse experimental procedures were conducted according to Institutional Animal Care and Use Committee (IACUC)-approved protocols. Female NSG mice were obtained from Charles River. To evaluate the safety and efficacy of SynNotch to CAR circuits, 6- to 8-week-old animals were inoculated with 5×10⁶ high He2-K562 cells and 5×10⁶ low Her2 K562 cells in PBS solution, subcutaneously in the right and left flanks, respectively. Single dose treatments consisting of sorted and rested 4.0×10⁶ CD4+ and 4.0×10⁶ CD8′ engineered or the matched number of untransduced T cells were administered intravenously via tail vein in 100 l of PBS at day 7 after tumor injection. Tumor volumes were monitored two times a week via caliper measurements until predetermined IACUC-approved endpoint (hunching, neurological impairments such as circling, ataxia, paralysis, limping, head tilt, balance problems, seizures, tumor volume burden) was reached (n=5 to 7 mice per group

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An antigen-density sensing molecular circuit comprising:
(a) a nucleic acid sequence encoding an antigen-triggered transcriptional switch that binds with low affinity to an antigen present on the surface of a target cell;
(b) a nucleic acid sequence encoding an antigen-specific therapeutic that binds with high affinity to the antigen; and
(c) a regulatory sequence operably linked to (b) that is activated by binding of the antigen-triggered transcriptional switch to the antigen to induce expression of the antigen-specific therapeutic.

2. The molecular circuit according to claim 1, wherein the target cell is a cancer cell.

3. The molecular circuit according to claim 2, wherein the antigen is selected from the group consisting of: Receptor tyrosine-protein kinase erbB-2 (HER2), CAMPATH-1 antigen (CD52), Programmed cell death 1 ligand 1 (PD-L1), Vascular endothelial growth factor (VEGF), B-lymphocyte antigen CD19 (CD19), Tumor necrosis factor receptor superfamily member 8 (CD30), Glutamate carboxypeptidase 2 (PSMA), Epidermal growth factor receptor (EGFR), disialoganglioside GD2 (GD2), SLAM family member 7 (SLAMF7), Myeloid cell surface antigen CD33 (CD33), B-lymphocyte antigen CD20 (CD20), B-cell receptor CD22 (CD22), Platelet-derived growth factor receptor alpha (PDGFRA), Vascular endothelial growth factor receptor 1 (VEGFR1), Vascular endothelial growth factor receptor 2 (VEGFR2), Mucin 1 (MCU1), Glutamate carboxypeptidase 2 (FOLH1), and Tyrosine-protein kinase receptor UFO (AXL).

4. The molecular circuit according to any of the preceding claims, wherein the antigen-specific therapeutic comprises a single antigen-binding domain specific for the antigen.

5. The molecular circuit according to any of claims 1 to 3, wherein the antigen-specific therapeutic comprises multiple antigen-binding domains specific for the antigen.

6. The molecular circuit according to any of the preceding claims, wherein the antigen-triggered transcriptional switch comprises a single antigen-binding domain specific for the antigen.

7. The molecular circuit according to any of claims 1 to 5, wherein the antigen-triggered transcriptional switch comprises multiple antigen-binding domains specific for the antigen.

8. The molecular circuit according to any of the preceding claims, wherein the antigen-specific therapeutic is a chimeric antigen receptor (CAR), a T cell receptor (TCR), or an antibody.

9. The molecular circuit according to any of the preceding claims, wherein the antigen-triggered transcriptional switch comprises a Notch force sensor cleavage domain.

10. The molecular circuit according to claim 9, wherein the antigen-triggered transcriptional switch is a synNotch polypeptide.

11. The molecular circuit according to any of claims 1 to 8, wherein the antigen-triggered transcriptional switch comprises a non-Notch force sensor cleavage domain.

12. The molecular circuit according to claim 11, wherein the non-Notch force sensor cleavage domain comprises a von Willebrand Factor (vWF) cleavage domain.

13. A cell genetically modified to comprise the molecular circuit of any of the preceding claims.

14. The cell of claim 13, wherein the cell is an immune cell.

15. The cell of claim 14, wherein the immune cell is a myeloid cell or a lymphoid cell.

16. The cell of claim 15, wherein the immune cell is a lymphoid cell selected from the group consisting of: a T lymphocyte, a B lymphocyte and a Natural Killer cell.

17. The cell of any of claims 13 to 16, wherein the antigen-specific therapeutic is expressed on the surface of the cell.

18. The cell of any of claims 13 to 16, wherein the antigen-specific therapeutic is secreted by the cell.

19. A method of making an antigen-density sensing molecular circuit, the method comprising:
obtaining a sequence encoding an antigen binding domain that binds to an antigen;
generating a modified antigen binding domain sequence encoding:
a high affinity modified antigen binding domain with increased affinity for the antigen as compared to the antigen binding domain; or
a low affinity modified antigen binding domain with decreased affinity for the antigen as compared to the antigen binding domain; and
generating a molecular circuit encoding an antigen-triggered transcriptional switch comprising the antigen binding domain that, when activated, induces expression of an antigen-specific therapeutic comprising the high affinity modified antigen binding domain; or
generating a molecular circuit encoding an antigen-triggered transcriptional switch comprising the low affinity modified antigen binding domain that, when activated, induces expression of an antigen-specific therapeutic comprising the antigen binding domain.

20. The method according to claim 19, wherein the antigen-specific therapeutic is a chimeric antigen receptor (CAR), a T cell receptor (TCR), or an antibody.

21. The method according to claims 19 or 20, wherein the antigen-triggered transcriptional switch comprises a Notch force sensor cleavage domain.

22. The method according to claim 21, wherein the antigen-triggered transcriptional switch is a synNotch polypeptide.

23. The method according to claims 19 or 20, wherein the antigen-triggered transcriptional switch comprises a non-Notch force sensor cleavage domain.

24. The method according to claim 23, wherein the non-Notch force sensor cleavage domain comprises a von Willebrand Factor (vWF) cleavage domain.

25. A method of inducing expression of a high affinity therapeutic specific to an antigen expressed by a target cell in a subject in need thereof, the method comprising:
administering to the subject a cell genetically modified to comprise a molecular circuit comprising an antigen-triggered transcriptional switch that binds with low affinity to the antigen, wherein binding of the antigen-triggered transcriptional switch to the antigen induces expression of the high affinity therapeutic in the subject.

26. The method according to claim 25, wherein the antigen is a cancer antigen and the target cell is a cancer cell.

27. The method according to claims 25 or 26, wherein the high affinity therapeutic is a chimeric antigen receptor (CAR), a T cell receptor (TCR), or an antibody.

28. The method according to any of claims 25-27, wherein the genetically modified cell is a cell according to any one of claims 13 to 18.

29. A method of activating an immune response to a target cell expressing an antigen in a subject;
the method comprising:
administering to the subject an immune cell genetically modified to comprise a molecular circuit comprising an antigen-triggered transcriptional switch that binds with low affinity to the antigen to induce expression of an antigen-specific therapeutic that binds with high affinity to the antigen to activate the immune response in the subject.

30. The method according to claim 29, wherein the molecular circuit comprises an antigen-density sensing molecular circuit according to any one of claims 1 to 12.

31. A method of treating a subject for a cancer expressing an antigen, the method comprising:
administering to the subject an effective amount of immune cells genetically modified to comprise a molecular circuit comprising an antigen-triggered transcriptional switch that binds with low affinity to the antigen to induce expression of an antigen-specific therapeutic that binds with high affinity to the antigen to activate an immune response in the subject, thereby treating the subject for the cancer.

32. The method according to claim 31, wherein the antigen is also expressed by non-cancer cells in the subject.

33. The method according to claims 31 or 32, wherein the effective amount of immune cells comprises an immune cell according to any one of claims 14 to 16.


SEQUENCE LISTING (9 sequences)

SEQ ID NO: 1 - protein, 21 aa, Artificial Sequence
  Synthetic sequence
MALPVTALLLPLALLLHAARP

SEQ ID NO: 2 - protein, 10 aa, Artificial Sequence
  Synthetic sequence
EQKLISEEDL

SEQ ID NO: 3 - protein, 20 aa, Artificial Sequence
  Synthetic sequence
GGAGCACTGTCCTCCGAACG

SEQ ID NO: 4 - protein, 43 aa, Artificial Sequence
  Synthetic sequence
EARKAIARVKRESKRIVEDLIMSCAQESAASEKISREAERLIR

SEQ ID NO: 5 - protein, 246 aa, Artificial Sequence
  Synthetic sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGS

SEQ ID NO: 6 - protein, 246 aa, Artificial Sequence
  Synthetic sequence
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGS

SEQ ID NO: 7 - protein, 246 aa, Artificial Sequence
  Synthetic sequence
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGVKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGS

SEQ ID NO: 8 - protein, 246 aa, Artificial Sequence
  Synthetic sequence
DIQMTQSPSSLSASVGDRVTITCRASQDVNTAVAWYQQKPGKAPKLLIYSASFLESGVPSRFSGSGSGTDFTLTISSLQPEDFATYYCQQHYTTPPTFGQGVKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGS

SEQ ID NO: 9 - protein, 15 aa, Artificial Sequence
  Synthetic sequence
GGGGSGGGGSGGGGS